(19) United States
(12) Patent Application Publication
(10) Pub. No.: US 2004/0116350 A1
(43) Pub. Date: Jun. 17, 2004

(54) Methods and compositions relating to hydrogen peroxide and superoxide production by antibodies

(76) Inventors: Paul Wentworth Jr, San Diego, CA (US); Anita D. Wentworth, San Diego, CA (US); Lyn H. Jones, Canterbury (GB); Kim D. Janda, La Jolla, CA (US); Richard A. Lerner, La Jolla, CA (US)

Correspondence Address: Schwegman Lundberg, Woessner & Kluth, PO Box 2938, Minneapolis, MN, 55402, US

(21) Appl. No.: 10/380,905

(22) PCT Filed: Sep. 17, 2001
(86) PCT No.: PCT/US01/29165

(51) Int. Cl. A61K 38/44; A61K 38/05; A61K 31/385; A61K 9/127; A61K 31/05

(52) U.S. Cl. 514/18; 514/27; 514/456; 514/474; 424/94.4; 514/731; 424/450; 514/440

(57) ABSTRACT

The invention relates generally to the field of immunology. More specifically, the invention relates the finding that antibodies can generate superoxide and hydrogen peroxide from singlet oxygen. Accordingly, methods and compositions able to increase or decrease oxidative stress are provided. Also provided are screening assays to identify agents that modulate the ability of a antibody to generate superoxide and hydrogen peroxide. Such agents can be used therapeutically to treat patients in need. Further, the invention provides methods to use antibodies in immunoassays.

### FIELD OF THE INVENTION

The invention relates to methods for the antibody-mediated generation of superoxide free radical from singlet oxygen. The invention also relates to the generation of hydrogen peroxide from singlet oxygen. Therapeutic methods are based upon both enhancing and inhibiting these processes. Screening methods relate to identifying modulators of antibody-mediated generation of hydrogen peroxide and superoxide free radical through the respective increase or decrease in detectable hydrogen peroxide or superoxide. The invention further relates to a simplified immunoassay based on detecting hydrogen peroxide. The invention also relates to therapeutic compositions that are engineered to increase the production of hydrogen peroxide and superoxide free radical as well as compositions that are engineered to prevent this production.

### BACKGROUND

A relevant biological basis relating to varying disease mechanisms and conditions is that of oxidative stress and the consequent production of free radicals that paradoxically are both beneficial and detrimental to cellular metabolism. Human metabolism is oxygen based. As such, the chemical reactions relating to oxidative processes play a central role in cellular homeostasis.

In the oxygen cascade, reactive oxygen species resulting from incomplete reduction of oxygen include among others the free radicals, superoxide radical (O₂⁻) and hydroxyl radical (OH^{●}), that have one or more unpaired electrons. Superoxide spontaneously reacts with itself in a dismutation reaction to form hydrogen peroxide (H₂O₂). The formed hydrogen peroxide, while not being a free radical, under certain situations, e.g., in the absence of catalase, becomes a cytotoxic oxidant through the formation of hydroxyl radical and hypochlorous acid (HOCl) (McCord, Amer. J. Med. 108:652659 (2000)). Intracellularly, most of the superoxide is generated as a result of mitochondrial respiration (McCord, Amer. J. Med. 108:652659 (2000)). At low superoxide concentration, the conversion to hydrogen peroxide is catalyzed by superoxide dismutase, a process that helps maintain a lower steady-state concentration of superoxide (Babior et al., Amer. J. Med. 109:33-34 (2000)).

Another highly reactive molecule involved in the oxygen cascade is singlet oxygen (¹O₂). Singlet oxygen results from irradiation by light of metal-free porphyrin precursors that are present in the skin of porphyria sufferers. Singlet oxygen is also generated by neutrophils and is thought to be responsible for damage created by phagocytes on their targets (Babior et al., Amer. J. Med. 109:33-34 (2000)). Based on its high reactivity with biomolecules, singlet oxygen has generally been considered to be an endpoint in the cascade of oxygen-scavenging agents.

The reactive nature of free radicals causes them to have both positive and negative effects on cells and on whole organisms. Methods to inhibit the negative effects of these reactive species would be tremendously beneficial in the treatment of many conditions. Also, methods to utilize the positive effects of these reactive species would be beneficial to control such things as cell proliferation and infection. Accordingly, methods and agents are needed to modulate the generation of free radicals and other reactive species.

### SUMMARY OF THE INVENTION

The invention provides methods for utilizing the newly discovered abilities of an antibody to reduce singlet oxygen to superoxide. This catalytic reaction ultimately results in the formation of hydrogen peroxide. The invention also provides methods to utilize antibodies to produce hydrogen peroxide from singlet oxygen by the oxidation of water. Hydrogen peroxide, under certain biological conditions, itself generates reactive molecules. Thus, the invention generally provides methods to inhibit and facilitate these processes depending on the desired outcome. The invention further relates to screening methods to identify agents that modulate the newly discovered antibody-mediated processes. The invention further contemplates an improved immunoassay format based on the direct detection of hydrogen peroxide that is produced by antibody catalyzed oxidation of water. The invention also provides an improved immunoassay based on hydrogen peroxide produced from antibody-generated superoxide in the presence of singlet oxygen. The invention also contemplates therapeutic compositions, preferably antibody compositions, that are engineered to exhibit increased or decreased oxidative function.

### BRIEF DESCRIPTION OF THE DRAWINGS

FIG. 1 illustrates the oxygen-dependent microbicidal action of phagocytes. The interconversion of ¹O₂ and O₂ ^{●-} is indicated and is an intrinsic ability to antibodies.

FIG. 2 illustrates the amplex red assay.

FIG. 3 shows the initial time course of H₂O₂ production in PBS (pH 7.4) in the presence (□) or absence (Δ) of murine monoclonal IgG EP2-19G2 (20 μM). Error bars show the range of the data from the mean.

FIG. 4 shows the fluorescent micrograph of a single crystal of murine antibody 1D4 Fab fragment after UV irradiation and H₂O₂ detection with the amplex red reagent.

FIG. 5 illustrates the (A)HP sensitization assay. Time course of H₂O₂ formation in PBS (pH 7.4) with HP (40 μM) and visible light, in the presence (O) or absence (♦) of 31127 (horse IgG, 20 μM). (B) Initial time course of H₂O₂ production with HP (40 μM) and visible light, in the presence of 31127 (horse IgG, 6.7 μM) with no additive in PBS (pH 7.4) (□) or NaN₃ in PBS (pH 7.4) (0, 100 μM) or in a D₂O solution of PBS (pH 7.4) (⋄). (C) Protein concentration (31127, horse IgG) versus rate of H₂O₂ formation. (D) Oxygen concentration on the rate of H₂O₂ generation with 31127 (horse IgG, 6.7 μM). All points are mean values of at least duplicate experimental determinations. Error bars are the range of experimentally measured values from the mean.

FIG. 6 is a bar graph showing the measured initial rate of H₂O₂ formation for a panel of proteins and comparison with antibodies (data from Table I). All points are mean values of at least duplicate experimental determinations. Error bars are the range of experimentally measured values form the mean. OVA, chick-egg ovalbumin; SOD, superoxide dismutase.

FIG. 7 shows (A) the rate of H₂O₂ formation by UV irradiation of horse IgG (6.7 μM) in PBS (pH 7.4). (B) simultaneous fluorescence emission of the horse IgG, measured at 326 nm (excitation=280 nm).

FIG. 8 shows H₂O₂ production. (A) Production of H₂O₂ by immunoglobulins and non-immunoglobulin proteins. Assays were performed by near-UV irradiation (312 nm, 800 μW cm⁻²) of individual protein samples (100 μL, 6.7 μM) in phosphate-buffered saline (PBS) [10 mM sodium phosphate, 150 mM NaCl (pH 7.4)] in a sealed glass vial on a transilluminator (Fischer Biotech) under ambient aerobic conditions at 20° C. Aliquots (10 μL) were removed throughout the assay. H₂O₂ concentration was determined by the amplex red method. Each data point is reported as the mean±SEM of at least duplicate measurements: [● polyclonal (poly) immunoglobulin (Ig) G, human; 0 polyIgG, horse; □ polyIgG, sheep; ∇ monoclonal (m) IgG (WD16G6), murine; ΔpolyIgM, human; ⋄ mIgG (92H2), murine; ▪ β-galactosidase (βgal); ▴ chick ovalbumin (OVA); ▾ α-lactalbumin (α-lact); ♦ bovine serum albumin (BSA)]. (B) Long-term production of H₂O₂ by sheep polyIgG (6.7 μM, 200 μL). Near-UV irradiation for 8 hours in PBS in a sealed well of a 96-well quartz plate. H₂O₂ concentration was measured as described in (A). (C) A solution of PCP-21H3, mIgG (murine) (6.7 μM, 200 μL), was irradiated in PBS in a sealed well of a 96 well quartz plate for 510 min. The H₂O₂ was assayed by the amplex red assay and then destroyed by addition of catalase (10 mg, 288 mU) immobilized on Eupergit C. The catalase was removed by filtration and the antibody solution re-irradiated for 420 min. Rate (−510 min)=0.368 μM min⁻¹ (r²=0.998); rate (511-930 min)=0.398 μM min⁻¹(r²=0.987). (D)

Determination of IC₅₀ of H₂O₂ on the photo-production of H₂O₂ by horse polyIgG. A solution of horse IgG (6.7 μM) was incubated with varying concentrations of H₂O₂ (0-450 μM) and the initial rate of H₂O₂ formation measured as described in (A). The graph is a plot of rate of H₂O₂ formation versus H₂O₂ concentration and reveals an IC., of 225 μM. (E) Long-term inhibition of antibody photo-production of H₂O₂ by H₂O₂ and complete reestablishment of activity. The assay involved an initial U.V. irradiation of horse polyIgG (6.7 mM in PBS pH 7.4) in the presence of H₂O₂ (450 μM) for 360 min.

The H₂O₂ was then removed by catalase (immobilized on Eupergit C) and the polyIgG sample was re-irradiated with UV light for a further 480 minutes. H₂O₂ formation throughout the assay was measured by the amplex red assay. (F) A solution of αβ-TCR (6.7 μM, 200 μL) was irradiated as described in (C) for periods of 360, 367 and 389 min. The H₂O₂ generated during each irradiation was assayed and destroyed as described in (C). Rate (0-360 min) 0.693 μM min⁻¹ (r=0.962). The curvature in the progress curve above 200 μM conforms to the expected inhibition by H₂O₂ (vide infra); rate (361-727 min) 0.427 μM mine (r²=0.987); rate (728-1117 min)=0.386 μM min⁻¹ (r²=0.991).

FIG. 9 illustrates the superposition of native 4C6 Fab (light blue and pink in a color photograph) and 4C6 Fab in the presence of H₂O₂ (dark blue and red in a color photograph) (A). The native 4C6 crystals were soaked for 3 minutes in 4 mM H₂O₂, and immediately flash frozen for data collection at SSRL BL 9-1. The overall structural integrity of the secondary and tertiary structure is clearly preserved in the presence of H₂O₂ (RMSD Cα=0.33 Å, side chain=0.49 Å). The RMSD was calculated in CNS. (B) High resolution x-ray structures show that Fab 4C6 is cross-reactive with benzoic acid Superposition of the 4C6 combining site with and without H₂O₂ demonstrates that even the side chain conformations within the binding site are preserved (light and dark colored side chains in a color photograph correspond to + and −H₂O₂ respectively). Moreover, clear electron density for the benzoic acid underscores that the binding properties of Fab 4C6 remain unaltered in 4 mM H₂O₂. The electron density map is a 2fₒ-f_{c} sigma weighted map contoured at 1.5σ, and the figures were generated in Bobscript.

FIG. 10 shows the absorbance spectra of horse polyclonal IgG measured on a diode array HP8452A spectrophotometer, Absₘₐₓ 280 nm (A). (B) Action spectra of horse polyclonal IgG, between 260 and 320 nm showing maximum activity of H₂O₂ formation at 280 nm. The assay was performed in duplicate and involved addition of an antibody solution [6.7 μM in PBS (pH 7.4)] to a quartz tube that was then placed in a light beam produced by a xenon arc lamp and monochromator of an SLM spectrofluorimeter for 1 hour. H₂O₂ concentration was measured by the amplex red assay.

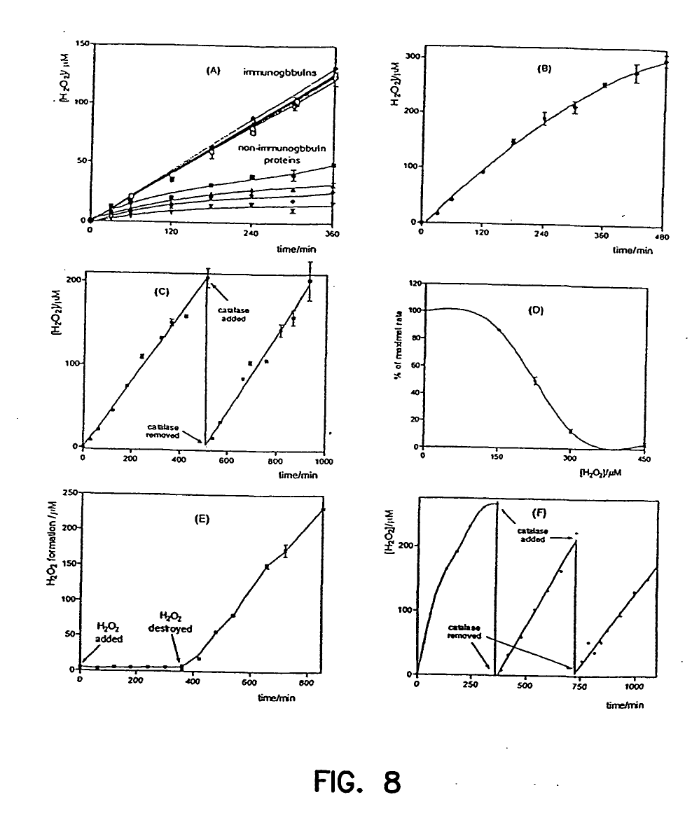

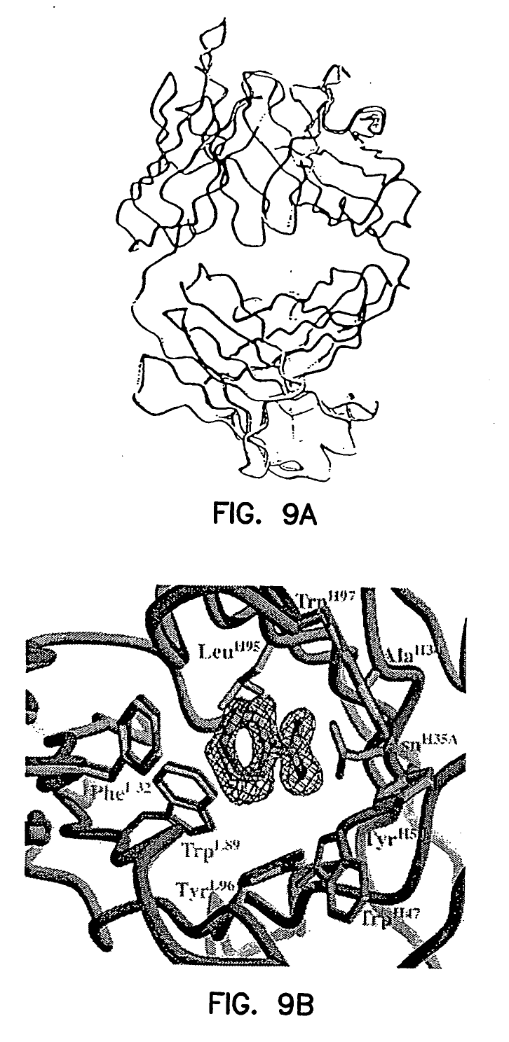

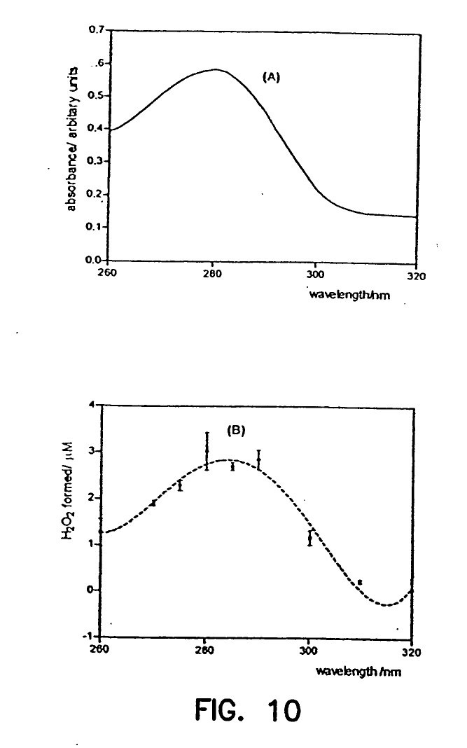

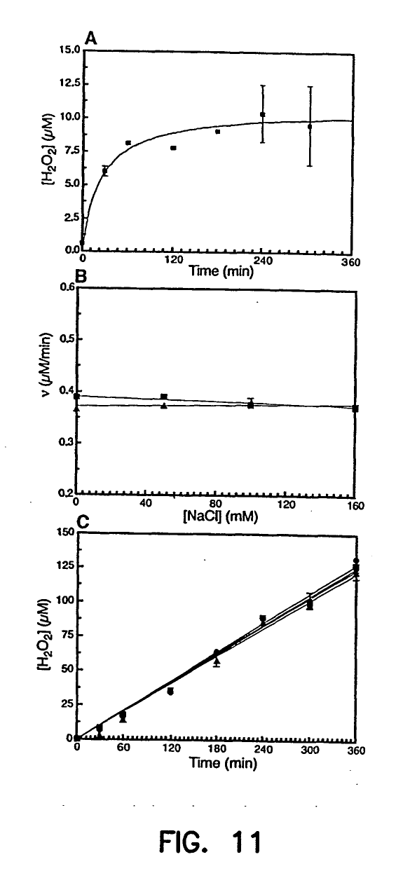

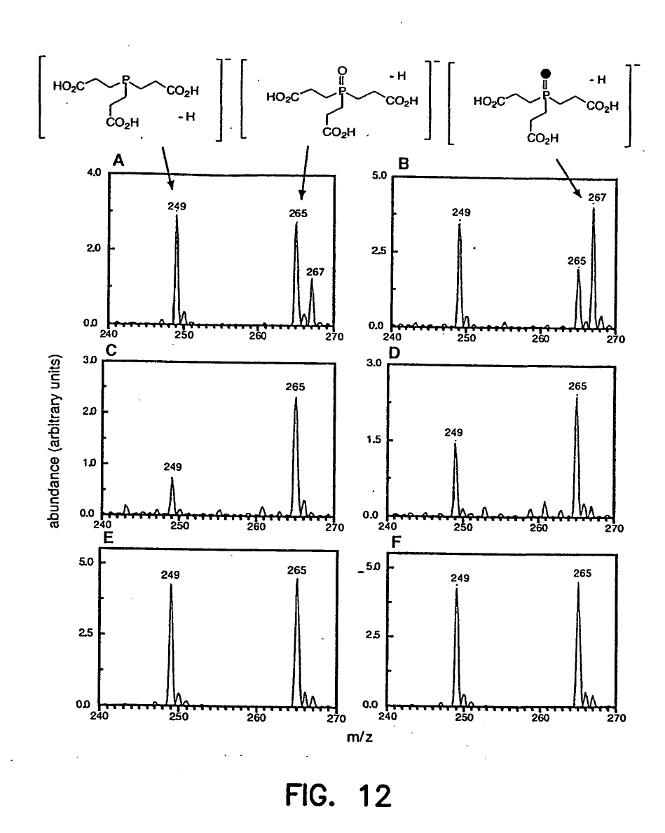

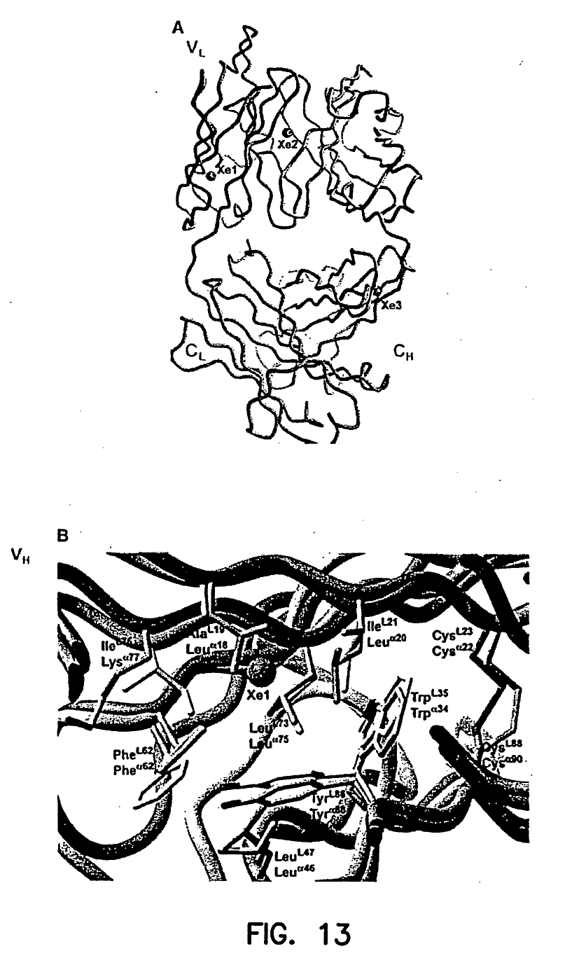

FIG. 11 shows the production of H₂O₂. (A) Production of H₂O₂ by tryptophan (20 μM). The conditions and assay procedures were as described in FIG. 8A. (B) Effect of chloride ion on antibody-mediated photo-production of H₂O₂. A solution of sheep polyIgG ▪ (6.7 μM, 200 μL) or horse polyIgG ▴ (6.7 μM, 200 μL) was lyophilized to dryness and then dissolved in either deionized water or NaCl (aq.) such that the final concentration of chloride ion were (0-160 mM). The samples were then irradiated, in duplicate, in sealed glass vials on a transilluminator (800 μW cm²) under ambient aerobic conditions at 20° C. Aliquots (10 μL) were removed throughout the assay and the H₂O₂ concentration determined by the amplex red assay. The rate of H₂O₂ formation is plotted as the mean±S.E.M. versus [NaCl] for each antibody sample. (C) Effect of dialysis into EDTA-containing buffers on antibody-mediated photo-production of H₂O₂. The photo-production of H₂O₂ by two antibody preparations, mouse monoclonat anitibody PCP21H3 sand horse polyclonal IgG, were compared before and after dialysis into PBS containing EDTA (20 mM). The conditions and assay procedures were as described in FIG. 8A. Each data point is reported as the mean±SEM of at least duplicate measurements: [● murine mIgG PCP21H3 before dialysis; ▪ murine mIgG PCP21H3 after dialysis; ▴ polyIgG, horse before dialysis; ♦ polyIgG, horse after dialysis.

FIG. 12 shows ESI (negative polarity) mass spectra of TCEP [(M−H) 249] and its oxide [(M−H) 265 (¹⁶O) and (M−H) 267 (¹⁸O)] produced by oxidation with H₂O₂. (A) MS of TCEP and its oxides after irradiation of sheep polyIgG (6.7/μM) under ¹⁶O₂ aerobic conditions in H₂ ¹⁸O (98% ¹⁸O) PB. (B) MS of TCEP and its oxides after irradiation of sheep polyIgG (6.7 μM) under enriched ¹⁸O₂ (90% ¹⁸O) aerobic conditions in H₂¹⁶O PB. (C) MS of TCEP and its oxides after irradiation of the polyIgG performed under ¹⁶O₂ aerobic concentration in H₂¹⁶O PB. The assay conditions and procedures were as described in the methods and materials (Example II) with the exception that H₂¹⁶ 0 replaced H₂¹⁸O. (D) MS of TCEP and its oxides after irradiation of sheep polyIgG (6.7 μM) and H₂¹⁶O₂ (200 μM) under anaerobic (degassed and under argon) conditions in H₂¹⁸O PB for 8 hours at 20° C. Addition of TCEP was as described in the methods and materials (Example II). (E) MS of TCEP and its oxides after irradiation of 3-methylindole (500 μM) under ¹⁶O₂ aerobic conditions in H₂¹⁸O PB. The assay conditions and procedures were as described in the methods and materials (Example II) with the exception that size-exclusion filtration was not performed because 3-methyl indole is of too low molecular weight. Therefore, TCEP was added to the 3-methyl indole-containing PB solution. (F) MS of TCEP and its oxides after irradiation of β-gal (50 μM) under ¹⁶O₂ aerobic conditions in H₂¹⁸O PB. Assay conditions and procedures are as described in the methods and materials (Example II).

FIG. 13 shows the Xe binding sites in antibody 4C6 as described in materials and methods (Example II). (A) Standard side view of the Cα trace of Fab 4C6 with the light chain in pink and the heavy chain in blue in a color photograph. Three bound xenon atoms (green in a color photograph) are shown with the initial Fₒ-F_{c} electron density map contoured at 5 σ. (B) Overlay of Fab 4C6 and the 2C αβ TCR (PDB/TCR) around the conserved xenon site 1. The backbone C_{α} trace of V_{L} (pink in a color photograph) and side chains (yellow in a color photograph) and the corresponding V_{α} of the 2C αβTCR (red and gold in a color photograph) are superimposed (figure generated using Insight2000).

### DETAILED DESCRIPTION OF THE INVENTION

The present invention concerns the discovery that antibodies, as a class of molecules, have an inherent capability to intercept singlet oxygen and convert it to either superoxide or hydrogen peroxide. This process acts to rescue and recycle oxygen, particularly during phagocyte-mediated processes, thereby contributing to microbicidal action of the immune system. These properties are common to all antibodies and were not known prior to the present invention. The common ability to convert singlet oxygen to superoxide or hydrogen peroxide, regardless of source or antigenic specificity, is thought to link the previously appreciated recognition properties of antibodies with killing events.

The present invention provides methods that relate to the ability of an antibody to reduce singlet oxygen (¹O₂) to superoxide radical (O₂⁻) and hydrogen peroxide. In view of the critical nature and role of oxygen metabolism in an aerobic organism, the identification of this biological process provides multiple and varied methods as described herein. The detailed determination and characterization of the antibody-mediated reduction of singlet oxygen is described in examples I and II.

As demonstrated in the examples, these properties are universal abilities of all antibodies.

Superoxide Production by Antibodies

The ability to produce superoxide from singlet oxygen is present in both intact immunoglobulins and well as Fab and F(ab′)₂ fragments (see examples). The activity does not reside in molecules, including RNaseA, superoxide dismutase, and Bowman-Birk inhibitor protein, that can be oxidized (example I and Table 1). Also, the activity is not associated with the presence of disulfides in a molecule, even though they are sufficiently electron rich that they can be oxidized (Bent et al., J. Am. Chem. Soc. 87:2612-2619 (1975)). Rather, the activity resides in an aromatic amino acid such as tryptophan that can be oxidized by singlet molecular oxygen via electron transfer (Grossweiner, Curr. Top. Radiat. Res. Q. 11:141-199 (1976)). The activity is further attributed to the indole component of the tryptophan residue. Thus, the indole acts as a reductive center in connection with the redox reaction. The indole portion becomes oxidized to form a radical cation in the course of reducing singlet molecule oxygen to superoxide free radical. In the same context, the antibody is called a reductant because it is oxidized in providing an electron to singlet molecular oxygen. It is believed that oxidized antibody interaction with an in vivo antioxidant completes the catalytic cycle and returns the antibody to neutrality. The ability of an antibody to generate superoxide from singlet oxygen is abolished if the antibody is denatured. This indicates that the location of the oxidized molecules in the reactive center of the antibody is relevant to the reduction process used to generate superoxide. In particular, the reduction of singlet molecular oxygen is primarily due to the two tryptophan residues that are buried in the molecule rather than the solvent-exposed ones (example I). Such buried aromatic residues in proteins, including antibodies, are generally considered to contribute to structure stability (Burley, et al., Science 229:23-28 (1985)). Furthermore, two aromatic tryptophan resides are conserved, referred to as TRP-36 and TRP47, and are both deeply buried (Kabat, et al., Sequences of Proteins of Immunological Interest, U.S. Department of Health and Human Services, Public Health Service, National Institutes of Health, Bethesda, Md. (1991)). The ability of antibodies as a class of proteins to reduce singlet molecular oxygen to superoxide anion is thus based on the presence of the conserved buried aromatic tryptophan residues.

Hydrogen Peroxide Production by Antibodies

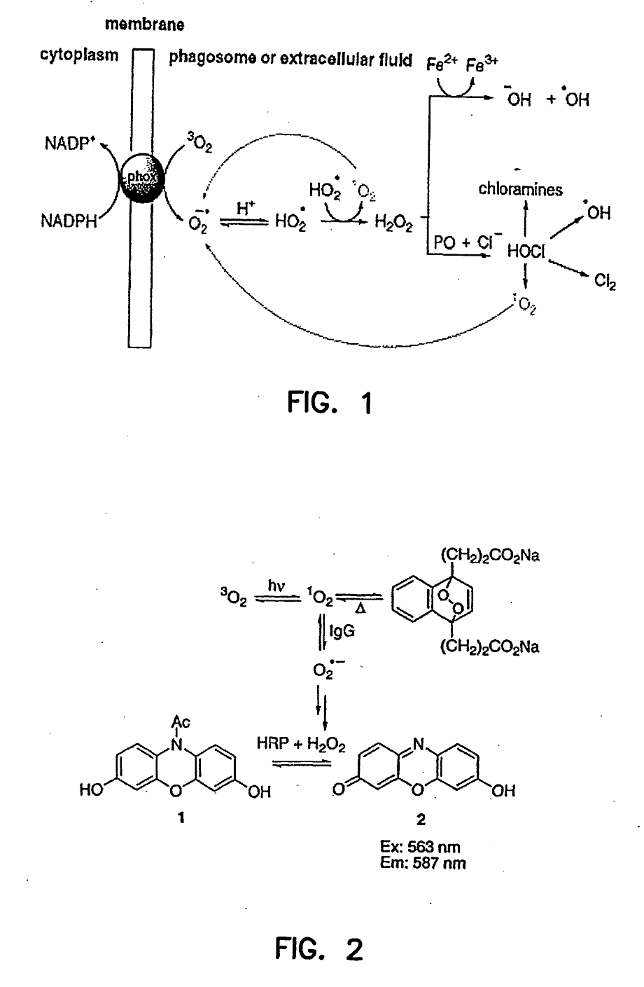

The ability to produce hydrogen peroxide in an efficient and long term manner from singlet oxygen is present in immunoglobulins and in the T-cell receptor (example II, FIG. 1F). The T-cell receptor shares a similar arrangement of its immunoglobulin fold domains with antibodies (Garcia et al., Science, 274:209 (1996)). However, possession of this structural motif does not appear necessary to confer a hydrogen peroxide-generating ability on proteins. β₂-macroglobulin, a member of the immunoglobulin superfamily having this structural motif, does not generate hydrogen peroxide (Welinder et al., Mol. Immunol. 28:177 (1991)). Structural studies suggest that a conserved tryptophan residue found in both the T-cell receptor and in antibodies may play a role in the oxidation of water. The catalytic role of the tryptophan conserved in antibodies and in the T-cell receptor is further supported by the observation that the β₂-macroglobulin lacks the conserved residue as well as the catalytic activity. Furthermore, the sequence and structure surrounding the conserved tryptophan residue is highly conserved between antibodies and the T-cell receptor indicating that it may also play a role in allowing catalysis of singlet oxygen to hydrogen peroxide.

Information relating the structure to the function of immunoglobulins and the T-cell receptor allows molecules to be designed that will catalyze the oxidation of water. This information also provides many new methods and treatment schemes that may be utilized based on existing molecules.

### Definitions

Abbreviations: (HP) hematoporphyrin; (PBS) phosphate buffered saline;

(OVA) chick-egg ovalbumin; (SOD) superoxide dismutase; (PO) peroxidase enzymes; (phox) phagocyte oxidase; (HRP) horseradish peroxidase; (MS) mass spectroscopy; (AES) ICP-atomic emission spectroscopy; (MS) mass-spectral, (QC) quantum chemical.

The term “agent” herein is used to denote a chemical compound, a mixture of chemical compounds, a biological macromolecule, or an extract made from biological materials such as bacteria, plants, fingi, or animal particularly mammalian) cells or tissues. Agents are evaluated for potential activity as antibody modulatory agents by inclusion in screening assays as described herein.

The term “antibody” as used in this invention includes intact molecules as well as fragments thereof, such as Fab, F(ab′)₂, and Fv which are capable of binding an epitope. These antibody fragments retain some ability to selectively bind with its antigen or receptor and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction;

F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“sFv”), defined as a genetically engineered molecule containing the variable region of the light chain, the variable region of the heavy chain, linked by a suitable polypeptide linker as a genetically fused single chain molecule.

The preparation of polyclonal antibodies is well-known to those skilled in the art See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology section 2.4.1 (1992), which are hereby incorporated by reference.

The preparation of monoclonal antibodies is also conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology Vol. 10, pages 79-104 (Humana Press (1992).

Methods of in vitro and in vivo manipulation of monoclonal antibodies are well known to those skilled in the art. One particular manipulation involves the process of humanizing a monoclonal antibody by recombinant means to generate antibodies containing human specific and recognizable sequences. See, for review, Holmes, et al., J. Immunol. 158:2192-2201 (1997) and Vaswani, et al., Annals Allergy Asthma & Immunol., 81:105-115 (1998).

Methods of making antibody fragments are known in the art (see for example, Harlow and Lane, Antibodies: A Laboratory Manual Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-inked by chemicals such as glutaraldehyde. Preferably, the Fv fragments comprise V_{α} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science, 242:423426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Lanick, et al., Methods: a Companion to Methods in Enzmology Vol. 2, page 106 (1991).

The terms “effective amount”, “effective reducing amount”, “effective ameliorating amount”, “effective tissue injury inhibiting amount”, “therapeutically effective amount” and the like terms as used herein are terms to identify an amount sufficient to obtain the desired physiological effect, e.g., treatment of a condition, disorder, disease and the like or reduction in symptoms of the condition, disorder, disease and the like. Such an effective amount of an antioxidant in the context of therapeutic methods is an amount that results in reducing, reversing, ameliorating, inhibiting, and the like improving directions, the effects of an oxidant generated by an antibody.

An “engineered molecule” is a polypeptide that has been produced through recombinant techniques. Such molecules can include a reactive center that can catalyze the production of superoxide or hydrogen peroxide from singlet oxygen. Such engineered molecules may have a reactive indole contained within a polypeptide structure. The indole of such a molecule may be present as a tryptophan residue. Engineered molecules may also contain non-natural amino acids and linkages as well as peptidomimetics. Engineered molecules also include antibodies that are modified to eliminate the reaction center such that they are no longer able to generate superoxide or hydrogen peroxide.

As used herein, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Antigens can include polypeptides, fatty acids, lipoproteins, lipids, chemicals, hormones and the like. In some embodiments, antigens include, but are not limited to, proteins from viruses such as human immunodeficiency virus, influenza virus, herpesvirus, papillomavirus, human T-cell leukemia virus and the lice. In other embodiments, antigens include, but are not limited to, proteins expressed on cancer cells such as lung cancer, prostate cancer, colon cancer, cervical cancer, endometrial cancer, bladder cancer, bone cancer, leukemia, lymphoma, brain cancer and the like. Antigens of the invention also include chemicals such as ethanol, tetrahydrocanabinol, LSD, heroin, cocaine and the like.

The term “modulate” refers to the capacity to either enhance or inhibit a functional property of an antibody or engineered molecule of the invention, such as production of superoxide or hydrogen peroxide.

A “non-natural” amino acid includes D-amino acids as well as amino acids that do not occur in nature, as exemplified by 4hydroxyproline, γ-carboxyglutamate, O-phosphoserine, N-acetylserine, N-formylmethionine, 3-methylhistidine, 5-hydroxylysine and other such amino acids and imino acids.

The term “peptidomiinetic” or “peptide mimetic” describes a peptide analog, such as those commonly used in the pharmaceutical industry as non-peptide drugs, with properties analogous to those of the template peptide. (Fauchere, J., Adv. Drug Res. 15: 29 (1986) and Evans et al., J. Med. Chem., 30:1229 (1987)). Generally, peptidomimetics are structurally similar to a paradigm polypeptide (i.e., a polypeptide that has a biochemical property or pharmacological activity), but have one or more peptide linkages optionally replaced by a linkage such as, —CH₂NH—, —CH₂S—, —CH₂—CH₂—, —CH═CH—(cis and trans), —COCH₂—, —CH(OH)CH₂—, and —CH₂SO—, by methods known in the art. Advantages of peptide mimetics over natural polypeptide embodiments may include more economical production, greater chemical stability, altered specificity, reduced antigenicity, and enhanced pharmacological properties such as half-life, absorption, potency and efficacy.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The terms “protein” and “polypeptide” are used to describe a native protein, fragments, or analogs of a polypeptide sequence. These terms may be used interchangeably.

Antibodies

The invention provides therapeutic antibodies. All antibody molecules belong to a family of plasma proteins called immunoglobulins. Their basic building block, the immunoglobulin fold or domain, is used in various forms in many molecules of the immune system and other biological recognition systems. A typical immunoglobulin has four polypeptide chains, contains an antigen binding region known as a variable region, and contains a non-varying region known as the constant region. An antibody contemplated for use in the present invention can be in any of a variety of forms, including a whole immunoglobulin, Fv, Fab, other fragments, and a single chain antibody that includes the variable domain complementarity determining regions (CDR), or other forms. All of these terms fall under the broad term “antibody” as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described herein, an antibody or fragment thereof is used that is immunospecific for an antigen.

The preparation of a therapeutic antibody of this invention can be accomplished by recombinant expression techniques as well as protein synthesis, methods of which are well known to one of ordinary skill in the art. For recombinant approaches, mutation of a nucleic acid that encodes an antibody or fragment thereof can be conducted by a variety of means, but is most conveniently conducted using mutagenized oligonucleotides that are designed to introduce mutations at predetermined sites that then encode an altered amino acid sequence in the expressed molecule. Such alterations include substitutions, additions, and/or deletions of particular nucleotide sequences that similarly encode substitutions, additions, and/or deletions of the encoded amino acid residue sequence. Site-directed mutagenesis, also referred to as oligonucleotide-directed mutagenesis and variations thereof, and the subsequent cloning of the altered genes are well known techniques (Sambrook et al., Molecular Cloning: A Laboratory Manual, 2nd ed., Chapter 15, Cold Spring Harbor Laboratory Press, (1989)). Another recombinant approach includes synthesizing the gene encoding a therapeutic molecule of this invention by combining long oligonucleotide strands that are subsequently annealed and converted to double-stranded DNA suitable for cloning and expression (Ausebel et al., Current Protocols in Molecular Biology. Units 10 and 15, Wiley and Sons, Inc. (2000)). Such techniques can be used to create engineered molecules that contain a reduction center and are able to generate hydrogen peroxide or superoxide from singlet oxygen. It is contemplated that such engineered molecules can be designed based on antibody structure and on the T-cell receptor, in the case of hydrogen peroxide.

Thus, the present invention contemplates an antibody that has been engineered to generate more superoxide free radical or hydrogen peroxide in a desired location. The antibody is engineered to contain additional reductive centers, as described in examples I and II herein, that increase the reduction of singlet molecular oxygen to superoxide free radical or hydrogen peroxide. The invention also contemplates an antibody that has been engineered to have at least a diminished capacity to generate superoxide free radical or hydrogen peroxide from singlet oxygen. In that context, the antibody lacks at least one of its reductive centers and preferably is substantially free of a reductive center. Such antibody compositions are readily prepared with methods well known to one of ordinary skill in the art.

If desired, polyclonal or monoclonal antibodies prepared for use as therapeutic compositions or in the methods of invention can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (Coligan, et al., Unit 9, Current Protocols in Immunology Wiley Interscience, (1991)).

1. Therapeutic Methods

Because aerobic organisms rely on oxygen metabolism in a chemical environment where the toxicity of oxygen and metabolites thereof are paramount consequence, these organisms have evolved a multitude of mechanisms to maintain homeostasis and the overall health of the organism. The toxic potential of oxygen is attributed to the formation, in vivo, of reactive free radicals. To become toxic, oxygen must be activated, a process that occurs either by photoactivation resulting in singlet oxygen production or by reduction followed by the formation of hydrogen peroxide and the hydroxyl radical. The latter process is accelerated by the presence of transition metals, such as iron and copper, and/or specific enzymes such as monooxygenase. These processes occur in cellular compartments including mitochondria, microsomes, peroxysomes and the cytoplasmic membrane. See, Sahnoun et al., Therapie 52:251-270 (1997).

The free radicals that result from oxygen activation are by definition chemical species that possess one or several mismatched electrons. Free radicals are generated when a single electron is removed from the molecule. This results in a molecule that has at least one of its electrons unpaired to another electron. The resultant free radical is reactive since it seeks out available electrons from other molecules, the process of which can create a second reactive molecule thereby setting off a chain reaction.

Free radicals, also referred to as oxidants herein include superoxide, hydroxyl radical, halogenated oxygens and nitrogen containing molecules. Superoxide radical generated from the antibody-mediated reduction of singlet oxygen is itself an oxidant and also provides for the production of hydrogen peroxide. The latter, which while not itself an oxidant or reactive molecule, can generate reactive oxygen species that include hydroxyl radical, its secondary products such as carbon, oxygen, nitrogen or sulfur, which can react with other compounds to produce yet other free radicals creating a free radical chain reaction. (Babior et al., Am. J. Med. 109:3344 (2000)). Other reactive species that are a consequence of the oxygen cascade include oxidized halogens, such as hypochlorous acid (HOCl), the HOCl-generated reactive species chloramine (NH₂Cl) and aldehydes, and reactive nitrogen species.

A potential consequence of uncontrolled reactivity of free radicals is damage to DNA, RNA, membrane lipids, lipoproteins or enzymes, ultimately affecting the body. An end result is poor cell function leading to disease and even tissue death. Paradoxically, free radicals aid the process of riding the body of unwanted bacteria or viruses. However, when the production of radicals is excessive or in the wrong location, acute and chronic cellular and tissue injury can occur.

To counteract these reactions, aerobic organisms have evolved certain built-in mechanisms to keep the equilibrium of oxygen metabolism in check. These mechanisms broadly include inhibition of oxygen activation processes as well as neutralization of free radicals already formed. Neutralizing processes include 1) enyzmes such as superoxide dismutase and catalase that together produce peroxidases and 2) molecules such as tocopherols, carotenoids, ubiquinones, flavonoids, ascorbic acid, uric acid and similar molecules that serve as a source of electrons that are provided to free radicals without damaging cellular components. Such processes are considered beneficial to the well being of an organism. For example, the authors of a recent paper have correlated an increase in life span in an animal with exposure to superoxide dismutase/catalase mimetics (Meloy et al., Science, 289:1567-1569 (2000)). In the context of the present invention, any molecule that inhibits the antibody mediated generation of hydrogen peroxide or superoxide that ultimately leads to hydrogen peroxide formation is referred to as an antioxidant. Such preferred antioxidants of this invention are described below.

When the balance of oxidants to antioxidants tips in favor of the former, the oxidative state is generally referred to as “oxidative stress”. This situation occurs in the presence of an excess production of oxidants or free radicals and a diminishing of the control antioxidat mechanisms. Advantages of the present invention are that the discovery of the role an antibody plays in the generation of oxidants in the oxygen cascade provides the basis for therapeutic methods that are useful in maintaining oxygen balance and control of oxygen metabolism, depending on the desired outcome. In other words, the methods of this invention provide 1) for the production of oxidants when their production is warranted, such as in promoting wound healing, lysing bacteria, eliminating viruses, targeting cancer cells for oxidant-induced lysis and the like processes, and 2) for the inhibition of antibody generated oxidants by exposure of antioxidants when the inhibition of antibody generated oxidants is warranted, such as in inflammation, heart conditions, diabetes and unwanted cellular proliferation. For example, one may want to use antibody mediated generation of superoxide or hydrogen peroxide to supplement the local concentration of superoxide concentration generated by phagocytic neutrophils to combat a bacterial infection in a wound Here the neutrophil that contains NADPH oxidase produces superoxide radical in the presence of molecular oxygen. The superoxide in effect acts as bactericidal agent destroying the bacteria and ultimately the neutrophil in the process. Thus, to enhance this process, one would use the method of this invention to provide an antibody composition to the area to cause an increase in the local concentration of superoxide. On the other hand, neutrophil-generated superoxide is deleterious in inflamed joints such as in patients with rheumatoid arthritis who are concomitantly undergoing intensive humoral antibody-mediated immune responses. In such conditions, one would want to employ the opposing therapeutic method of this invention in providing an antioxidant to control the production of damaging oxidants produced by both neutrophils and antibodies in the local environment The decision to use the methods of this invention to inhibit or promote the antibody-mediated generation of superoxide and hydrogen peroxide and their derivatives (i.e., molecules derived therefrom) products and/or their effects is thus dependent on the desired outcome.

A. Inhibiting Antibody Activity

According to the invention, certain therapeutic methods for affecting “antibody mediated production of hydrogen peroxide” have been developed. Thus, the term “antibody mediated production of hydrogen peroxide” encompasses the reactive species that are both precursor and derivative to the generation of hydrogen peroxide.

The use of molecules that effect the antibody mediated production of hydrogen peroxide is applicable to any situation in which unwanted, deleterious, damaging production of reactive oxidant species that are generated by antibodies. The molecules that are useful in these situations are referred to generally as “antioxidants”, defined as any molecule that has an antagonist effect to an oxidant. An antioxidant so defined includes 1) inhibitors of an antibody thereby inhibiting superoxide generation, 2) inhibitors of hydrogen peroxide generation, 3) inhibitors of the reactions converting hydrogen peroxide into derivative reactive oxidants; and 4) inhibitors of the reactive oxidants themselves. Preferred antioxidants include those that inhibit the activation of oxygen producing reactive oxidants as well as those neutralizing those already formed. The antioxidant effect can occur by any mechanism, including catalysis. Antioxidants as a category include oxygen scavengers or free radical scavengers. Antioxidants may be of different types so they are available if and when they are needed. In view of the presence of oxygen throughout an aerobic organism, antioxidants may be available in different cellular, tissue, organ and extracellular compartments. The latter include extracellular fluid spaces, intraocular fluids, synovial fluid, cerebrospinal fluid, gastrointestinal secretions, interstitial fluid, blood and lymphatic fluid. Antioxidants are present within an organism but are also provided by supplementing the diet and in the methods of this invention. Particularly preferred antioxidants include but are not limited to ascorbic acid, α-tocopherol, γ-glutamylcysteinylglycine, γ-glutamyl transpeptidase, α-lipoic acid, dihydrolipoate, -acetyl-5-methoxytryptamine, flavones, flavonenes, flavanols, catalase, peroxidase, superoxide dismutase, metallothionein, and butylated hydroxytoluene. A further preferred molecule that has the capacity to function as an antioxidant in the context of the methods of this invention is an engineered antibody in which the ability to generate superoxide free radical from reducing singlet oxygen is diminished or preferably absent altogether. Such antibody molecules are described herein.

The use of antioxidants is directed to situations in which an antioxidant is required to prevent, control, minimize, reduce, or inhibit the damage of an oxidant. Thus, the invention contemplates the use of an antioxidant for reducing the antibody mediated production of hydrogen peroxide in a cell. In such situations, without intervention, the cellular damage may be so extensive that tissue injury results, for example, in inflammatory conditions, in trauma conditions, in organ transplantation and the like. In the context of using an engineered antibody as an antioxidant, the antibody, having diminished or substantially no ability to generate superoxide or hydrogen peroxide since it lacks the reductive centers that reduce singlet oxygen, provides a therapeutic benefit in promoting a desired immune response without inducing additional tissue damage resulting from excess superoxide production. Preferred engineered therapeutic antibody compositions retain their antigen binding site so that targeting to a particular antigen is achieved in concert with the desired therapeutic benefits.

The present invention further contemplates a method of ameliorating oxidative stress in a subject as well as alleviating a symptom in a subject where the symptom is associated with production of oxidant. Exemplary of conditions in which the therapeutic methods of inhibiting the antibody mediated production of hydrogen peroxide with an antioxidant of the present invention include but are not limited to inhibiting aberrant smooth muscle disorder, inhibiting liver disease, proliferation of cancer cells, inhibiting inflammation in cancer patients receiving radiotherapy, inflammatory diseases (arthritis, vasculitis, glomerulonephritis, systemic lupus erythematosus, and adult respiratory distress syndrome), ischemic diseases (heart disease, stroke, intestinal ischemia, and reperfilsion injury), hemochromatosis, acquired inmmunodeficiency syndrome, emphysema, organ transplantation, gastric ulcers, hypertension, preeclampsia, neurological diseases (multiple sclerosis, Alzheimer's disease, Parkinson's disease, amyotrophic lateral sclerosis, and muscular dystrophy) alcoholism and smoking-related diseases.

Cells in which oxidative stress is deleterious include but are not limited to endothelial, interstitial, epithelial, muscle (smooth, skeletal or cardiac), phagocytic (including neutrophils and macrophages), white blood cells, dendritic, connective tissue and nervous system cells. Effected tissues include but are not limited to muscle, nervous, skin, glandular, mesenchymal, splenic, sclerous, epithelial and endothelial tissues.

The literature as well as patented inventions describe the use of antioxidants and oxygen scavengers to treat various conditions induced by oxidative stress, other than that relating to the generation of oxidants by an antibody as described in the present invention. Thus, the disclosures of U.S. Pat. Nos. 5,362,492; 5,599,712; 5,637,315; 5,647,315; 5,747,026; 5,848,290; 5,994,339; 6,030,611 and 6,040,611 support the therapeutic uses of antioxidants in the present invention, and as such, the disclosures of which patents are hereby incorporated by reference.

The oxidants and oxygen scavengers of the invention may be formulated into a variety of acceptable compositions. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, -ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts are obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids also are made.

The oxidants and oxygen scavengers may be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the oxidants and oxygen scavengers may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of oxidants and oxygen scavengers in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the oxidants and oxygen scavengers in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the oxidants and oxygen scavengers plus any additional desired ingredient present in the previously sterile-filtered solutions.

For topical administration, the oxidants and oxygen scavengers may be applied in pure form, i.e., when they are liquids. However, it will generally be desirable to administer them to the skin as compositions or formulations, in combination with a dermatologically acceptable carrier, which may be a solid or a liquid

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the oxidants and oxygen scavengers of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the oxidants and oxygen scavengers of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the oxidants and oxygen scavengers of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the oxidants and oxygen scavengers, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The oxidants and oxygen scavengers are conveniently administered in unit dosage form; for example, containing 5 to 1000 mg, conveniently 10 to 750 mg, most conveniently, 50 to 500 mg of active ingredient per unit dosage form.

Ideally, the oxidants and oxygen scavengers should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the oxidants and oxygen scavengers, optionally in saline, or orally administered as a bolus containing about 1-100 mg of the oxidants and oxygen scavengers. Desirable blood levels may be maintained by continuous infusion to provide about 0.01-5.0 mg/kg/hr or by intermittent infusions containing about 0.415 mg/kg of the oxidants and oxygen scavengers.

The desired dose may conveniently be presented in a single dose or as divided doses administered at appropriate intervals, for example, as two, three, four or more sub-doses per day. The subdose itself may be further divided, e.g., into a number of discrete loosely spaced administrations; such as multiple inhalations from an insufflator or by application of a plurality of drops into the eye.

In a preferred embodiment, an antioxidant enters the cell and reacts with the hydrogen peroxide or its precursor oxygen molecules thereby reducing the hydrogen peroxide concentration in the cell. In an alternative embodiment, an antioxidant enters the cell or is present in the surrounding extracellular milieu and reacts with the oxidants generated from hydrogen peroxide.

The therapeutic compositions of this invention, the antioxidants described herein, antibodies that include both engineered antibodies and other molecules containing additional reductive centers as described herein for promoting antibody activity, are administered in a manner compatible with the dosage formulation, and in a therapeutically effective amount The quantity to be administered and timing depends on the subject to be treated, capacity of the subject's system to utilize the active ingredient, and degree of therapeutic effect desired. Precise amounts of active ingredient required to be administered depend on the judgement of the practitioner and are peculiar to each individual. However, suitable dosage ranges for various types of applications depend on the route of administration. Suitable regimes for administration are also variable, but are typified by an initial administration followed by repeated doses at intervals to result in the desired outcome of the therapeutic treatment.

Antioxidants contemplated for use in the present invention are delivered to the site of interest to mediate the desired outcome in a composition such as a liposome, the preparation of which is well known to one of ordinary skill in the art of liposome-mediated delivery. Alternative delivery means include but are not limited to administration intravenously, topically, orally, by inhalation, by cannulation, intracavitally, intramuscularly, transdermally, and subcutaneously.

Therapeutic compositions of the present invention contain a physiologically tolerable carrier together with an antioxidant as described herein or an antibody as described herein for providing antibody activity, dissolved or dispersed therein as an active ingredient. In a preferred embodiment, the therapeutic composition is not immunogenic when administered to a mammal or human patient for therapeutic purposes.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic compositions of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

Other therapeutic conditions that would benefit from the antioxidant inhibition of antibody mediated oxidant production in a cell, tissue, or organs as well as extracellular compartments are well known to those of ordinary skill in the art and have been reviewed by McCord, Am. J. Med. 108:652-659 (2000) and Babior et al., Am. J. Med. 109:3344 (2000), the disclosures of which are hereby incorporated by reference.

B. Providing Antibody Activity

The present invention also generally contemplates the use of any antibody to generate superoxide radical or hydrogen peroxide in a situation where the production of superoxide or hydrogen peroxide is warranted. The present invention also contemplates the use of engineered molecules including engineered antibodies that have been altered to contain a reductive center, the presence of which provides for the capability to generate superoxide or hydrogen peroxide from singlet oxygen when such production is desired In the case of superoxide, the use of engineered molecules having more than two reductive centers compared to a non-engineered antibody having the two conserved tryptophan residues is warranted when enhanced production of superoxide is needed. Thus, for the therapeutic methods that benefit from a production of superoxide free radical, also called superoxide, the present invention contemplates the use of antibodies as defined above that contain the naturally occurring buried tryptophan residues as well as the engineered antibodies and other molecules described herein. In the case of hydrogen peroxide, the use of engineered molecules having additional reductive centers is warranted when enhanced production of hydrogen peroxide is needed. Thus, for the therapeutic methods that benefit from a production of hydrogen, the present invention contemplates the use of antibodies as defined above that contain naturally occurring tryptophan residue as well as the engineered antibodies and other molecules described herein.

The conditions under which hydrogen peroxide or superoxide radical and its consequent production of hydrogen peroxide is generated by an antibody is more completely described in examples I and II. The minimum requirement for generating hydrogen peroxide or superoxide is the presence of oxygen, i.e., aerobic conditions. The biological reduction of singlet oxygen to hydrogen peroxide or superoxide radical that results in hydrogen peroxide occurs both visible light and ultraviolet irradiation conditions. In the former, the production of hydrogen peroxide is enhanced in the presence of photosensitizer molecules such as hematoporphyrin. Moreover, ultraviolet light irradiation is not essential for the antibody mediated reduction events. In the absence of light, antibody mediated production of superoxide or hydrogen peroxide occurs when aerobic conditions are present along with a superoxide or hydrogen peroxide generating amount of photosensitizer.

In view of the minimal requirements for the antibody mediated generation of hydrogen peroxide or superoxide that results in hydrogen peroxide production, the present invention contemplates the therapeutic use of an antibody to create an superoxide or hydrogen peroxide environment where one does not exist or enhance an already existing one. Such conditions are well known to practitioners in the art of oxygen cascade chemistry and the generation of oxidants to provide a desired beneficial outcome such as those described herein.

In one embodiment, the invention contemplates a method for exposing an antigen to superoxide and hydrogen peroxide where the antigen is contacted with a composition including an antibody able to generate hydrogen peroxide or superoxide from singlet oxygen. As previously discussed, the method is successful with either nonspecific or immunospecific (antigen directed) intact antibody, fragments derived therefrom and further including single chain antibodies as well as the engineered molecules and antibodies described herein. Exemplary concentrations of antibody at the cell surface range from 1 to 5 micromolar. However, the concentration may vary depending on the desired outcome where the amount of antibody provided is that amount of antibody that is sufficient to obtain the desired physiological effect, i.e, the generation of hydrogen peroxide or superoxide radical and its derivative oxidants to generate oxidative stress. Dosing and timing of the therapeutic treatments with antibody compositions are compatible with those described for antioxidants above. The antigen is preferably presented on a cell but need not be so limited. The antigen can be any antigen that is present in a cell, tissue or organ including extracellular fluids where the presence of superoxide and the antibody mediated process of producing it is warranted. In a preferred embodiment, the antigen is a fatty acid, a low density lipoprotein, an antigen associated with inflammation, a cancer cell antigen, a bacterial antigen or a similar molecule.

Cells on which antigens are associated include but are not limited to endothelial, interstitial, epithelial, muscle, phagocytic, blood, dendritic, connective tissue and nervous system cells. Particularly preferred target cells for the present therapeutic approach are neutrophils or macrophages.

The invention further contemplates exposing a target cell to irradiation with either ultraviolet, infrared or visible light in the method of generating antibody superoxide or hydrogen peroxide.

To enhance the production of superoxide or hydrogen peroxide, a superoxide or hydrogen peroxide generating amount of a photosensitizer, also referred to as a sensitizer, is utilized in the therapeutic methods described herein. As defined herein, a sensitizer is any molecule that induces or increases the concentration of singlet oxygen. Sensitizers are generally used in the presence of irradiation, the process of which includes exposure to ultraviolet, infrared or visible light for a period sufficient to activate the sensitizer. Exemplary exposures are described in examples I and II. A superoxide or hydrogen peroxide generating amount of sensitizer is the amount of sensitizer that is sufficient to obtain the desired physiological effect, e.g., generation of superoxide or hydrogen peroxide from singlet oxygen mediated by an antibody in any situation where superoxide or hydrogen peroxide presence and the derivatives thereof is warranted. In a preferred embodiment, a sensitizer is conjugated to the antibody. In a particularly preferred embodiment, a sensitizer conjugated antibody is capable of binding to a antigen, i.e., retains an active antigen binding site, allowing for antigen recognition and complexing to occur. Exemplary sensitizers include but are not limited to pterins, flavins, hematoporphyrin, tetrakis(4-sulfonatophenyl)porphyrin, bipyridyl ruthemium(II) complexes, rose bengal dye, quinones, rhodamine dyes, phtalocyanine, and hypocrellins.

In a further embodiment, the generation of superoxide or hydrogen peroxide is enhanced by administering a means to enhance the production of singlet oxygen. Reduced singlet oxygen is the source of superoxide or hydrogen peroxide as previously discussed. Such reduction can occur through the action of an antibody or molecule containing greater than two reductive centers. One preferred means is referred to as a prodrug that is any molecule, compound, reagent and the like that is useful in generating singlet oxygen. A preferred prodrug is endoperoxide, that is administered at a time subsequent to the administering or contacting of an antibody with a desired target cell, tissue or organ as described below. In this context, endoperoxide is preferably delivered after a superoxide or hydrogen peroxide producing antibody or molecule has immunoreacted with its target antigen forming an antibody-antigen complex. A preferred concentration of endoperoxide to achieve at the antibody-antigen complex site is about 10 micromolar. This embodiment has particular advantages. For example, the ability to create an increased local accumulation of singlet oxygen provides the necessary reactant to be reduced to the therapeutically desirable superoxide or hydrogen peroxide at a desired site or location.

Preferred therapeutic methods based on the use of an antibody including an engineered antibody or molecule having reductive centers to generate superoxide or hydrogen peroxide from singlet oxygen includes a method for killing a cancer cell where the cancer cell is contacted with a composition including an antibody capable of generating superoxide or hydrogen peroxide from singlet oxygen. In a preferred embodiment, the antibody recognizes and immunoreacts with an antigen expressed on the cancer cell. Such methods are therapeutically useful for a subject with lung cancer, prostate cancer, colon cancer, cervical cancer, endometrial cancer, bladder cancer, bone cancer, leukemia, lymphoma, or brain cancer. In one aspect, the cancer cell is removed from a subject with cancer and cultured ex vivo for exposing to an antibody, and can further be exposed to ultraviolet light, infrared light or visible light for the cell to then be returned to the subject.

In other aspects, the antibody composition is delivered in vivo to a subject with cancer. Preferred in vivo delivery methods include administration intravenously, topically, by inhalation, by cannulation, intracavitally, intramuscularly, transdermally, subcutaneously or by liposome containing the antibody.

In still further aspects, the antibody is a recombinant antibody, that is provided as above or alternatively is expressed from an expression vector delivered to the cell. The expression vector in this context can also express a sensitizer molecule.

Therapeutic compositions in pharmaceutically acceptable excipients and pharmaceutically effective amounts as described for antioxidant containing compositions are applicable to the use of antibody containing compositions.

Additional therapeutic methods based on using an antibody that is able to generate superoxide or hydrogen peroxide from singlet oxygen are 1) for inhibiting proliferation of a cancer cell, 2) for targeting and killing a cancer cell in a patient where the antibody recognizes and immunoreacts with an antigen expressed on the cancer cell, 3) for inhibiting tissue injury associated with neutrophil mediated inflammation in a subject, for example where the inflammation results from a bacterial infection or when the subject has an autoimmune disease, 4) for enhancing the bactericidal effectiveness of a phagocyte in a subject, 5) for promoting wound healing in a subject having a open wound where the superoxide or hydrogen peroxide stimulates fibroblast proliferation and/or the immune response that further includes lymphocyte proliferation, 6) for stimulating cell proliferation, such as stimulating fibroblast proliferation in a wound in a subject, and the like situations. For wound healing, topical application to a wound on a subject is a preferable delivery approach such as with a bandage containing an antibody. Other therapeutic conditions that would benefit from the creation or enhancement of superoxide or hydrogen peroxide in a cell, tissue, organ or extracellular compartment are well known to those of ordinary skill in the art and have been reviewed by McCord, Am. J. Med. 108:652659 (2000), the disclosure of which are hereby incorporated by reference.

2. Screening Methods

The invention further contemplates screening methods that are based on the newly discovered antibody reduction of singlet oxygen to hydrogen peroxide or superoxide radical.

Thus, in one embodiment the invention contemplates a method for identifying an agent that modulates antibody mediated production of hydrogen peroxide or superoxide. A modulator is a molecule that either inhibits or promotes the production of superoxide or hydrogen peroxide. Either type of modulator is identifiable with the same method. In a preferred embodiment, the method includes the steps of:

a) contacting a composition comprising an antibody capable of generating superoxide or hydrogen peroxide with an agent to form an admixture in an assay solution in the presence of molecular oxygen;

b) irradiating the admixture to generate singlet oxygen from molecular oxygen, wherein the singlet oxygen is reduced to hydrogen peroxide or superoxide by the antibody, wherein the superoxide dismutates to form hydrogen peroxide;

c) detecting the formed hydrogen peroxide; and

d) comparing the detected hydrogen peroxide with a suitable control, thereby determining how the agent modulates the production of hydrogen peroxide or superoxide.

The irradiating step is performed with either ultraviolet light or visible light. With the latter form, a sensitizer as previously described can be added with the antibody composition.

The formed hydrogen peroxide is detected through reaction directly with a hydrogen peroxide where the reacted substrate is detected with a fluorescent means, such as with fluorescent microscopy or fluorescent spectrometry. In fluorescent spectrometry, detection is ELISA based or with done with a standard cuvette. Exemplary assay methods are performed as described in examples I and II.

In a separate screening method of the present invention, a method for performing an immunoassay to detect antibody immunoreactivity with an antigen is also contemplated based on the discovery of antibody generated superoxide or hydrogen peroxide. The method comprises the steps of.

a) contacting in a singlet oxygen-generating medium a substrate having immobilized thereon a composition comprising a first reagent comprising an antigen or an antibody, with a second composition comprising an antigen or an antibody that is reactive with the first reagent to form an immobilized antigen-antibody complex, wherein the antibody generates superoxide or hydrogen peroxide from singlet oxygen in the presence of oxygen; and

b) detecting the antibody-generated superoxide or hydrogen peroxide, thereby detecting the antibody immunoreactivity with the antigen.

The reaction and detection means are those as described herein. In one aspect, the first composition is an antigen and the second composition is an antibody. In the opposite aspect, the first composition is an antibody and the second composition is an antigen.

The invention further contemplates a similar method for performing an immunoassay to detect antibody immunoreactivity with an antigen where an antigen is immobilized and contacted with an antibody composition.

Such immunoassay methods are an improvement over those that are well known as methods to assess antigen-antibody immunoreactivity and to identify antigens and/or antibodies. The advantage of the present method over previous other immunoassay methods lies in the present elimination of at least one method step and/or the incorporation of a secondary labeled immunoreactive molecule, the labeling either being a radioactive or enzymatic compound.

In the present invention, the minimum requirements are oxygen, an antibody reagent, an antigen reagent, and a detectable reactant that reacts with hydrogen peroxide generated from the antibody. A preferred reactant is a fluorogenic substrate. One such reactant used as described in examples I and II is called AMPLEX™ Red. It is a commercially available reagent sold by Molecular Probes (Eugene, Oreg.) for reacting antibody generated hydrogen peroxide in the immunoassay. It is sold in a kit that provides a one-step fluorometric method for measuring hydrogen peroxide using a fluorescent microplate or fluorimeter for detection. The assay is based on the detection of hydrogen peroxide using 10-acetyl-3,7-dihyroxyphenoxazine, a highly sensitive and stable probe for hydrogen peroxide.

In the presence of horseradish peroxidase, the AMPLEX™ Red reagent reacts with hydrogen peroxide in a 1:1 stoichiometry to produce highly fluorescent resorufin, that provides a detection mechanism to detect as little as 10 picomoles of hydrogen peroxide in a 200 microliter volume.

In contrast, prior immunoassay techniques, including radioimmunoassays (RIA), enzyme-immunoassays (EIA), and the classic enzyme-linked immunosorbent assay (ELISA), all require either the use of a radioactively labeled immunoreactive molecule as in RIA or an additional labeled immunoreactive molecule. The present invention neither requires potentially harmful radioactive isotopes to label a molecule or requires an additional immunoreactive reagent that generally is referred to as a secondary antibody that is usually conjugated with an enzyme to allow for the detection of the complex formed with the first antibody with the antigen. In the latter assays, the reaction of the secondary antibody with the formed antigen-antibody complex (generally through an anti-first antibody specificity immunoreactivity) is detected through a color-producing substrate solution specific for the conjugated enzyme. In summary, in the present invention, the antibody mediated generation of hydrogen peroxide is detected with high detection capacity without radioactive agents, without requiring an additional reagent and/or admixing step such as those practiced in U.S. Pat. Nos. 3,905,767; 4,016,043; USRE032696; and 4,376,110, the disclosures of which are hereby incorporated by reference.

3. Therapeutic Compositions

The present invention contemplates therapeutic compositions useful in practicing the therapeutic methods as described above. Antibodies, as a class of proteins, are now known to act as reductants in reducing singlet molecular oxygen (also referred to herein as singlet oxygen) to generate superoxide free radical (also referred to herein as superoxide). As a result of the redox reaction, the antibody becomes oxidized. The oxidation of the antibody is now known to occur at the two buried tryptophan residues as further discussed in example I. The activity is further ascribed to the indole component of the tryptophan residue. Thus, in view of the redox reaction where the indole portion becomes oxidized forming a radical cation in the reaction of reducing singlet molecule oxygen to superoxide free radical, the indole is referred to as a reductive center. A reductive center as defined in the present invention as having the ability to reduce singlet oxygen to superoxide and becoming oxidized in the process. Preferably, a reductive center is more efficient if it is not solvent-exposed, i.e., is buried within the therapeutic composition defined herein.

Therapeutic compositions may also be produced and used according to the therapeutic methods described above with antibodies and engineered molecules that produce hydrogen peroxide through oxidation of water. Antibodies, as a class of proteins, are now known to catalyze the oxidation of water to produce hydrogen peroxide. The activity is ascribed to a conserved tryptophan residue.

Thus, the present invention contemplates therapeutic compositions that are useful in either acting to reduce the local concentration of hydrogen peroxide or superoxide production or in the alternative useful in acting to enhance it. Such compositions contain reagents referred to generally as being “engineered”, defined herein to connote a reagent, such as an antibody or fragment thereof as defined herein, or other molecule, that has been altered in some form to either increase or decrease the number of reductive centers as defined herein.

The invention thus contemplates an antibody that has been engineered to have at least a diminished capacity to generate hydrogen peroxide or superoxide free radical from singlet oxygen. In that context, the antibody lacks at least one of its reductive centers and preferably is substantially free of a reductive center. Such antibody compositions are readily prepared with recombinant expression methods well known to one of ordinary skill in the art. In preferred embodiments, the antibody retains the same amino acid residue number but the reductive center has been replaced or substituted with a component that lacks the ability to reduce singlet oxygen In such aspects, the reductive center comprises a buried indole and preferably, in the case of superoxide, two buried indoles. In particularly preferred embodiments, the reductive center comprises an indole on a tryptophan residue that is substituted by another amino acid that does not have reductive capacity. Such preferred substitutions includes the amino acids phenylalanine and alanine. In other aspects, the present invention also contemplates deletion of the tryptophan without replacement or substitution thereof as long as the desired antibody activity, particularly antigen binding activity, is not adversely affected. As previously discussed, an engineered antibody having reduced or absent reductive centers while retaining antigen targeting ability provides the therapeutic advantage of providing an antibody to stimulate a desired immune response in particular situations while reducing or eliminating altogether the undesirable production of hydrogen peroxide or superoxide and its byproducts that can further damage cells and tissues. Methods for making an engineered antibody that functions as an antioxidant in the context of the therapeutic methods described herein are well known in the art, such as site-directed mutagenesis of a nucleotide sequence encoding the antibody of interest as previously discussed.

Engineered antibodies that function as an antioxidant according to the methods of the invention are contemplated for any of the methods as described herein.

The present invention also contemplates engineered therapeutic molecules including engineered antibodies that have been altered to contain a reductive center where they were in an insufficient amount to effect adequate production of superoxide or hydrogen peroxide, or where they are needed to increase the number of reductive centers to a number in excess of those that were naturally occurring in the molecule or antibody. Introduction of a reductive center in a engineered molecule or antibody is accomplished by methods well known to one of ordinary skill in the art Preferred means including recombinant expression methods and well as direct protein synthesis methods have been previously describe. The choice of method is necessarily dependent on the length of the molecule being engineered. Regardless of the methods employed, the positioning, i.e., the location, of the engineered reductive center is based upon the ability of the engineered molecule to exhibit reducing activity on singlet oxygen. Preferably, the incorporation of reductive centers are positioned such that they are deeply buried in the folded molecule allowing a retention of structural ability without comprising superoxide or hydrogen peroxide production. In one embodiment, in an antibody where it is desired to retain antigen binding function, the location of an engineered reductive center is adjacent to a variable binding domain. In certain aspects, one reductive center is contemplated. In other aspects, two reductive centers are contemplated. Still, in other aspects, more than three reductive centers are contemplated. Preferably, the reductive centers comprise indole. Also contemplated are reductive centers comprising indole present in tryptophan residue. Any technique to engineer such reductive centers in a molecule or antibody is contemplated for use in the present invention. In a preferred embodiment, the reductive centers are introduced by site-directed mutagenesis of nucleotide sequences encoding the engineered antibody such that the substituted nucleotides encode tryptophan residues at predetermined locations in the encoded molecule.

In the embodiment of preparing an engineered molecule such as an antibody to include desired reductive centers, such molecule that is produced by recombinant technology is also contemplated to be in the form of a fusion conjugate, where the conjugate provides a sensitizer molecule as previously described for use in therapeutic methods as described herein.

Engineered antibodies or other molecules, which can be any protein or polypeptide such that they contain reductive centers that function according to the methods of the invention, are contemplated for any of the methods as described herein.

The invention is further described in detail by reference to the non-limiting examples that follow. While the invention has been described in detail with reference to certain preferred embodiments thereof, it will be understood that modifications and variations are within the spirit and scope of that which is described and claimed.

### Example I

### Antibodies have the Intrinsic Capacity to Destroy Antigens

### Materials and Methods

Antibodies: The following whole antibodies were obtained from PharMingen: 49.2 (mouse IgG_{2b} κ), G155-178 (mouse IgG₂ₐκ), 107.3 (mouse IgG₁ κ), A95-1 (rat IgG₂κ), G235-2356 (hamster IgG), R3-34 (rat IgG κ), R35-95 (rat IgG₂. κ), 27-74 (mouse IgE), A110-1 (rat IgG₁ λ), 145-2C11 (hamster IgG group I κ), M18-254 (mouse IgA κ), and MOPC-315 (mouse IG λ). The following were obtained from Pierce: 31243 (sheep IgG), 31154 (human IgG), 31127 (horse IgG), and 31146 (human IgM).

The following F(ab′)₂ fragments were obtained from Pierce: 31129 (rabbit IgG), 31189 (rabbit IgG), 31214 (goat IgG), 31165 (goat IgG), and 31203 (mouse IgG). Protein A, protein G, trypsin-chymotrypsin inhibitor (Bowman-Birk inhibitor), β-lactoglobulin A, α-lactalbumin, myoglobin, β-galactosidase, chicken egg albumin, aprotinin, trypsinogen, lectin (peanut), lectin (Jacalin), BSA, superoxide dismutase, and catalase were obtained from Sigma. Ribonuclease I A was obtained from Amersham Pharmacia. The following immunoglobulins were obtained in-house using hybridoma technology: OB2-34C12 (mouse IgG₁ κ), SHO1-41G9 (mouse IgG₁ κ), OB3-14F1 (mouse IgG₃ κ), DMP-15G12 (mouse IgG₂ₐκ), AD1-19G1 (mouse IgG_{2b} κ), NTJ-92C12 (mouse IgG₁ κ), NBA-5G9 (mouse IgG₁ κ), SPF-12H8 (mouse IgG(3 κ), TIN-6C11 (mouse IgG. κ), PRX-1B7 (mouse IgG₂. κ), HA5-19A11 (mouse IgG₂. κ), EP2-19G2 (mouse IgG₁ κ), GNC-92H2 (mouse IgG₁ κ), WD1-6G6 (mouse IgG₁ κ), CH2-5H7 (mouse IgG_{2b} κ), PCP-21H3 (mouse IgG₁ κ), and TM1-87D7 (mouse IgG₁ κ). DRB polyclonal (human IgG) and DRB-b12 human IgG) were supplied by Dennis R. Burton (Ihe Scripps Research Institute). 1D4 Fab (crystallized) was supplied by Ian A. Wilson (The Scripps Research Institute).

All assays were carried out in PBS (10 mM phosphate/160 mM sodium chloride, pH 7.4). Commercial protein solution samples were dialyzed into PBS as necessary. Amplex Red hydrogen peroxide assay kits (A-12212) were obtained from Molecular Probes.

Antibody/Protein Irradiation. Unless otherwise stated, the assay solution (100 μl, 6.7 μM protein in PBS, pH 7.4) was added to a glass vial, sealed with a screw-cap, and irradiated with either UV (312 nm, 8000 μWcm² Fischer-Biotech transilluminator) or visible light.

Quantitative Assay for Hydrogen Peroxide. An aliquot (20 μl) from the protein solution was removed and added into a well of a 96-well microtiter plate (Costar) containing reaction buffer (80 μl). Working solution (100 μl/400 μM Amplex Red reagent ½ units/ml horseradish peroxidase) was then added, and the plate was incubated in the dark for 30 min. The fluorescence of the well components was then measured using a CytoFluor Multiwell Plate Reader (Series 4000, PerSeptive Biosystems, Framingham, Mass.; Ex/Em: 530/580 nm). The hydrogen peroxide concentration was determined using a standard curve. All experiments were run in duplicate, and the rate is quoted as the mean of at least two measurements.

Sensitization and Quenching Assays. A solution of 31127 (100 μl of horse IgG, 6.7 μM) in PBS (pH 7.4, 44% dimethylformamide) and hematoporphyrin IX (40 μM) was placed in proximity to a strip light. Hydrogen peroxide concentration was determined as described herein. The assay was also performed in the presence of NaN₃ (100 mM) or PBS in D₂O.

Oxygen Dependence. A solution of 31127 (1.6 ml, horse IgG, 6.7 μM) in PBS (pH 7.4) was rigorously degassed using the freeze/thaw method under argon. Aliquots (100 pi) were introduced into septum-sealed glass vials that had been purged with the appropriate O₂/Ar mixtures (0-100%) via syringe. Dissolved oxygen concentrations were measured with an Orion 862A dissolved-oxygen meter. These solutions were then vortexed vigorously, allowed to stand for 20 min, and then vortexed again. A syringe containing the requisite O₂/Ar mixture was used to maintain atmospheric pressure during the course of the experiment. Aliquots (20 μl) were removed using a gas-tight syringe and hydrogen peroxide concentration measured as described herein The data from three separate experiments were collated and analyzed using the Enzyme Kinetics v1.1 computer program (for determination of Vₘₐₓ and Kₘ parameters).

Antibody Production of Hydrogen Peroxide in the Dark, Using a Chemical ¹O₂ Source. A solution of sheep IgG 31243 (100 μl, 20 μM) in PBS (pH 7.4) and the endoperoxide of disodium 3,3′-1,4-naphthylidene) dipropionate (25 mM in D₂O) was placed in a warm room (37° C.) for 30 min in the dark. Hydrogen peroxide concentration was determined as described herein.

Hydrogen Peroxide Formation by the Fab1D4 Crystal. A suspension of crystals of the Fab fragment of 1D4 (2 μl) was diluted with PBS (198 μl, pH 7.4) and vortexed gently. Following centrifugation, the supernatant was removed, and the washing procedure was repeated twice further. The residual crystal suspension was then diluted into PBS, pH 7.4 (100 μl), and added into a well of a quartz ELISA plate. Following UV irradiation for 30 min, Amplex Red working solution (100 μl) was added, and the mixture was viewed on a fluorescence microscope.

Antibody Fluorescence Versus Hydrogen Peroxide Formation. A solution of −31127 (1.0 ml of horse IgG, 6.7 μM) in PBS (pH 7.4) was placed in a quartz cuvette and irradiated with UV light for 40 min. At 10-min intervals, the fluorescence of the solution was measured using an SPF-500C spectrofluorimeter (SLM-Aminco, Urbana, Ill.; Exim, 280/320). At the same time point, an aliquot (20 μl) of the solution was removed, and the hydrogen peroxide concentration was determined as described herein.

Consumption of Hydrogen Peroxide by Catalase. A solution of EP2-19G12 (100 μl of mouse IgG, 20 μM in PBS, pH 7.4) was irradiated with UV light for 30 min, after which time the concentration of hydrogen peroxide was determined by stick test (EM Quant Peroxide Test Sticks) to be 2 mg/liter. Catalase [1 μl, Sigma, 3.2 M (NH₄)₂SO₄, pH 6.0] was added, and after 1 min, the concentration of H₂O₂ was found to be 0 mg/liter.

Denaturation. IgG 19G12 (100 μl, 6.7 μM) was heated to 100° C. in an Eppendorf tube for 2 min. The resultant solution was transferred to a glass, screw-cap vial and irradiated with UV light for 30 min. The concentration of H₂O₂ was determined after 30 min.

### Results and Discussion

Research throughout the last century has led to a consensus as to the strategy of the humoral component of the immune system. The essence is that, for killing, the antibody molecule activates additional systems that respond to antibody-antigen union. It is now reported that the immune system has a previously unrecognized chemical potential intrinsic to the antibody molecule itself. All antibodies studied, regardless of source or antigenic specificity, can convert molecular oxygen into hydrogen peroxide, thereby potentially aligning recognition and killing within the same molecule. Aside from pointing to a new chemical arm for the immune system, these results are thought to be important to the understanding of how antibodies evolved and what role they may play in human diseases.

The antibody is a remarkable adaptor molecule, having evolved both targeting and effector functions that place it at the frontline of vertebrate defense against foreign invaders (Burton, D. R., Trends Biochem. Sci., 15, 6469 (1990)). In terms of the effector mechanism, the central idea is that antibodies themselves do not possess destructive ability but mark foreign substances for removal by the complement cascade and/or phagocytosis (Arlaud et al., Immunol. Today, 106-111 (1987); Sim & Reid, Immunol. Today. 12, 307-311 (1991)).

The advent of antibody catalysis has demonstrated that antibodies are capable of much more complex chemistry than simple binding (Wentworth & Janda, Curr. Opin. Chem. Biol. 2, 138-144 (1998)). This has inevitably led to the question as to whether more sophisticated chemical mechanisms are part of the strategy of the antibody molecule itself. Thus far, there has been no evidence to support this idea, and we are left with the notion that just because antibodies are capable of complex chemistry, it does not mean that they use it in host defense. However, it is now reported that a hitherto unremarked capacity of antibodies to convert molecular oxygen into hydrogen peroxide, thereby effectively linking recognition and killing events.

The preliminary step in the phagocytic oxidative burst is the single electron reduction of ground-state molecular oxygen (O₂) by the NADPH-dependent transmembrane phagocyte oxidase enzyme system that generates superoxide anion (O₂^{●-}) (FIG. 1) (Klebanoff, S. J. in Encyclopedia of Immunology, eds. Delves, P. J. & Roitt, I. M. (Academic, San Diego), pp. 1713-1718 (1998); Rosen, H. & Klebanoff, S. J., J. Biol. Chem, 252, 4803-4810(1997)).

Superoxide anion occupies a critical position in the cycling of oxygen dependent microbicidal agents iii vivo because although it is not itself considered to be cytotoxic (Fee, J. A. in International Conference on Oxygen and Oxygen-Radicals eds. Rodgers, M. A. J. & Powers, E. L. (Academic, San Diego, and University of Texas at Austin), pp. 205-239 (1981)), it is a direct precursor of hydrogen peroxide and the toxic derivatives it spawns, such as hydroxyl radical (HO^{●}) and hypochlorous acid (HOCl). In addition, when iron concentrations are limiting, O₂^{●-} is a vital reducing agent that regenerates Fe²⁺, thus facilitating the iron-catalyzed Haber-Weiss reaction, or the so-called superoxide-driven Fenton reaction that produces HO^{●} (Esq. 1 and 2). Therefore, processes that facilitate the generation of O₂^{●-} will ultimately perpetuate and potentiate oxygen-dependent microbicidal action.

Fe³⁺+O₂^{●-}→Fe²⁺+O₂ [1]

Fe²⁺+H₂O₂→Fe³⁺+⁻OH+HO^{●} [2]

Another key component of the oxygen-scavenging cascade is singlet molecular oxygen (¹O₂). This particularly reactive species is an excited state of molecular oxygen in which both outer shell electrons are spin-paired (Kearns, D. R., Chem. Rev. 71, 395427 (1971)). It is important in pathological biological systems and has a very short life-time (ca. 4 tis) in vivo (Foote, C. S. in Free Radicals in Biology, ed. Pryor, W. A. (Academic, New York), pp. 85-133 (1976)). Generation of ¹O₂ during microbicidal processes is either direct, via the action of flavoprotein oxidases (Allen, R C., Stjernholm, R. L., Benerito, K R & Steele, R. H., eds. Cormier, M. J., Hercules, D. M. & Lee, J. (Plenum, New York), pp. 498499 (1973); Klebanoff, S. J. in The Phagocvtic Cell in Host Resistance (National Institute of Child Health and Human Development, Orlando, Fla.) (1974)), or indirect, via the nonenzymatic disproportionation of O₂^{●-} in solutions at low pH, as found in the phagosome (Eq. 3) (Stauff, J., Sander, U. & Jaeschke, W., Chemiluminescence and Bioluminescence eds., Williams, R. C. & Fudenberg, H. H. (Intercontinental Medical Book Corp., New York), pp. 131-141 (1973); Allen, R C., Yevich, S. J., Orth, P, W. & Steele, R. H., Biochem. Biophys. Res. Commun. 60, 909-917 (1974)).

O₂^{●-}+2HO^{●}₂→¹O₂+H₂O₂ [3]

The high reactivity of ¹O₂ with biomolecules has meant that it is generally considered to be an endpoint in the cascade of oxygen-scavenging agents. However, it has been found that antibodies, as a class of proteins, have the intrinsic ability to intercept ¹O₂ and efficiently reduce it to O₂^{●-}, thus offering a mechanism by which oxygen can be rescued and recycled during phagocyte action, thereby potentiating the microbial action of the immune system.

The measured values for the initial rate of formation of hydrogen peroxide by a panel of intact immunoglobulins and antibody fragments are collected in Table 1. It is believed that Ig-generated O₂^{●-} dismutates spontaneously into H₂O₂, which is then utilized as a cosubstrate with N-acetyl-3,7-dihydroxyphenazine I (Amplex Red) for horseradish peroxidase, which produces the highly fluorcscent resorufin 2 (excitation maxima 563 nm, emission maxima 587 nm) (FIG. 2) (Zhou, M., Diwu, Z., Panchuk-Voloshina, N. & Haugland, R. P., Anal. Biochem., 253, 162-168 (1997)). To confirm that irradiation of the buffer does not generate O_{2*}- and that the antibodies are not simply acting as protein dismutases (Petyaev, I. M. & Hunt, J. V., Redox Report. 2, 365-372 (1996)), the enzyme superoxide dismutase was irradiated in PBS. Under these conditions, the rate of hydrogen peroxide generation is the same as irradiation of PBS alone.

TABLE 1
Production of hydrogen peroxide* by immunoglobulins

| | | | | Rate,^{†} |
|---|---|---|---|---|
| Entry | Clone | Source | Isotype | nmol/min/mg |
| 1 | CH25H7 | Mouse | IgG2b, κ | 0.25 |
| 2 | WD16G6 | Mouse | IgG1, κ | 0.24 |
| 3 | SHO-141G9 | Mouse | IgG1, κ | 0.26 |
| 4 | OB234C12 | Mouse | IgG1, κ | 0.22 |
| 5 | OB314F1 | Mouse | IgG2a, κ | 0.23 |
| 6 | DMP15G12 | Mouse | IgG2a, κ | 0.18 |
| 7 | AD19G1 | Mouse | IgG2b, κ | 0.22 |
| 8 | NTJ92C12 | Mouse | IgG1, κ | 0.17 |
| 9 | NBA5G9 | Mouse | IgG1, κ | 0.17 |
| 10 | SPF12H8 | Mouse | IgG2a, κ | 0.29 |
| 11 | TIN6C11 | Mouse | IgG2a, κ | 0.24 |
| 12 | PRX1B7 | Mouse | IgG2a, κ | 0.22 |
| 13 | HA519A4 | Mouse | IgG1, κ | 0.20 |
| 14 | 92H2 | Mouse | IgG1, κ | 0.41 |
| 15 | 19G2 | Mouse | IgG1, κ | 0.20 |
| 16 | PCP-21H3 | Mouse | IgG1, κ | 0.97 |
| 17 | TM1-87D7 | Mouse | IgG1, κ | 0.28 |
| 18 | 49.2 | Mouse | IgG2b, κ | 0.24 |
| 19 | 27-74 | Mouse | IgE, std. isotype | 0.36 |
| 20 | M18-254 | Mouse | IgA, κ | 0.39 |
| 21 | MOPC-315 | Mouse | IgA, λ | 0.39 |
| 22 | 31203 | Mouse | F(ab′)₂ | 0.21 |
| 23 | b12 | Human | IgG | 0.45 |
| 24 | polyclonal | Human | IgG | 0.34 |
| 25 | 31154 | Human | IgG | 0.18 |
| 26 | 31146 | Human | IgM | 0.22 |
| 27 | R3-34 | Rat | IgG1, κ | 0.27 |
| 28 | R35-95 | Rat | IgG2a, κ | 0.17 |
| 29 | A95-1 | Rat | IgG2b | 0.15 |
| 30 | A110-1 | Rat | IgG1, λ | 0.34 |
| 31 | G235-2356 | Hamster | IgG | 0.24 |
| 32 | 145-2C11 | Hamster | IgG, gp 1, κ | 0.27 |
| 33 | 31243 | Sheep | IgG | 0.20 |
| 34 | 31127 | Horse | IgG | 0.18 |
| 35 | polyclonal | Horse | IgG | 0.34 |
| 36 | 31229 | Rabbit | F(ab′)₂ | 0.19 |
| 37 | 31189 | Rabbit | F(ab′)₂ | 0.14 |
| 38 | 31214 | Goat | F(ab′)₂ | 0.24 |
| 39 | 31165 | Goat | F(ab′)₂ | 0.25 |

*Assay conditions are described in Materials and Methods.
^{†}Mean values of at least two determinations. The background rate of H₂O₂ formation is 0.005 nmol/min in PBS and 0.003 nm/min in PBS with SOD.

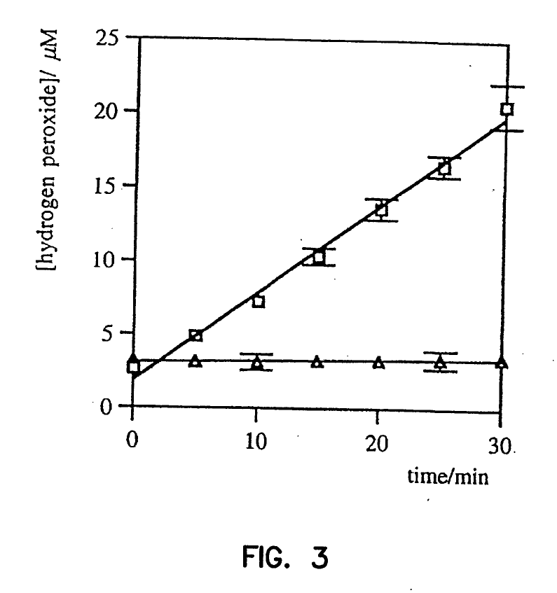

The rates of hydrogen peroxide formation were linear for more than 10% of the reaction, with respect to the oxygen concentration in PBS under ambient conditions (275 μM). With sufficient oxygen availability, the antibodies can generate at least 40 equivalents of H₂O₂ per protein molecule without either a significant reduction in activity or structural fragmentation. An example of the initial time course of hydrogen peroxide formation in the presence or absence of antibody 19G2 is shown in FIG. 3A. This activity is lost following denaturation of the protein by heating.

The data in Table I reveal a universal ability of antibodies to generate H₂O₂ from ¹O₂. This function seems to be shared across a range of species and is independent of the heavy and light chain compositions investigated or antigen specificity. The initial rates of hydrogen peroxide formation for the intact antibodies is highly conserved, varying from 0.15 nmol/min/mg [clone A95-1 (rat IgG2b)] to 0.97 nmol/min/mg (clone PCP-21H3, a murine monoclonal IgG) across the whole panel. Although the information available is more limited for the component antibody fragments, the activity seems to reside in both the Fab and F(ab′)₂ fragments.

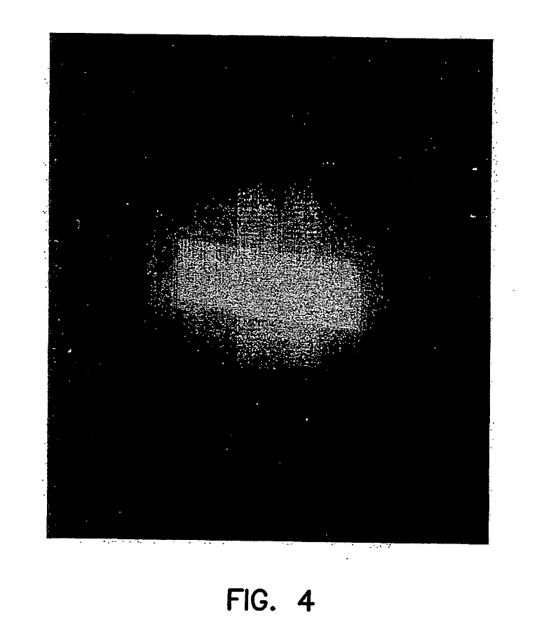

If this activity were due to a contaminant, it would have to be present in every antibody and antibody fragment obtained from diverse sources. However, to further rule out contamination, crystals of the murine antibody 1D4 Fab from which high-resolution x-ray structures have been obtained (at 1.7 Å) were investigated for their ability to generate H₂O₂ (FIG. 4). Reduction of ¹O₂ is clearly observed in these crystals.

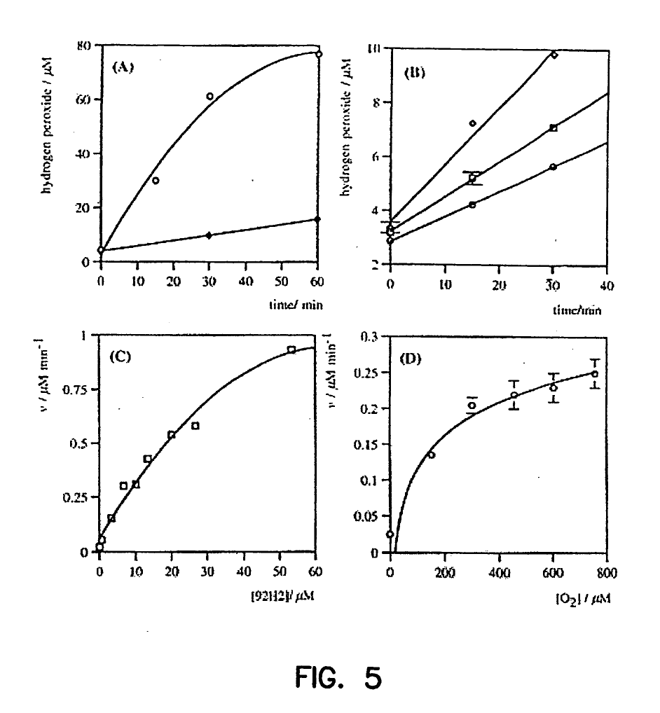

Investigations into this antibody transformation support singlet oxygen as the intermediate being reduced. No formation of hydrogen peroxide occurs with antibodies under anaerobic conditions either in the presence or absence of UV irradiation. Furthermore, no generation of hydrogen peroxide occurs under ambient aerobic conditions without irradiation. Irradiation of antibodies with visible light in the presence of a known photosensitizer Of 302 in aqueous solutions (Kreitner, M., Alth, (3., Koren, H., Loew, S. & Ebermann, R., Anal. Biochem. 213, 63-67 (1993)), hematoporphyrin (HP), leads to hydrogen peroxide formation (FIG. 5A). The curving in the observed rates is due to consumption of oxygen from within the assay mixture. Concerns that the interaction between photoexcited HP and oxygen may be resulting in O₂^{●-} formation (Beauchamp, C. & Fridovich, I., Anal. Biochem. 44, 276-287 (1971); Srinivasan, V. S., Podolski, D., Westrick, N. J. & Neckers, D. C., J. Am. Chem. Soc. 100, 6513-6515 (1978)) were largely discounted by suitable background experiments with the sensitizer alone (data shown in FIG. 5A). The efficient formation of H₂O₂ with HP and visible light both reaffirm the intermediacy of ¹O₂ and show that TV radiation is not necessary for the Ig to perform this reduction.

Furthermore, incubation of sheep antibody 31243 in the dark at 37° C., with a chemical source of ¹O₂ [the endoperoxide of 3′,3′-(1,4-naphthylidene)dipropionate] leads to hydrogen peroxide formation.

The rate of formation of H₂O₂, by horse IgG with HP (40 μM) in visible light, is increased in the presence of D₂O and reduced with the ¹O₂ quencher NaN₃ (40 mM) (FIG. 5B) (Hasty, N., Merkel, P. B., Radlick, P. & Kearns, D. R Tetrahedron Lett. 49-52 (1972)). The substitution of D₂O for H₂O is known to promote ¹O₂-mediated processes via an increase of approximately 10-fold in its lifetime (Merkel, P. B., Nillson, R. & Kearns, D. R., J. Am. Chem. Soc. 94, 1030-1031 (1972)).

The rate of hydrogen peroxide formation is proportional to IgG concentration between 0.5 and 20 μM but starts to curve at higher concentrations (FIG. 5C). The lifetime of ¹O₂ in protein solution is expected to be lower than in pure water due to the opportunity for reaction. It is therefore thought that the observed curvature may be due to a reduction in the lifetime of ¹O₂ due to reaction with the antibody.

Significantly, the effect of oxygen concentration on the observed rate of H₂O₂ production shows a significant saturation about 200 μM of oxygen (FIG. 5D). Therefore, the mechanism of reduction may involve either one or more oxygen binding sites within the antibody molecule. By treating the raw rate data to nonlinear regression analysis and by fitting to the Michaelis-Menten equation, a Kₘapp(O₂) of 187 μM and a V_{M}app of 0.4 nmol/min/mg are obtained. This antibody rate is equivalent to that observed for mitochondrial enzymes that reduce molecular oxygen in vivo.

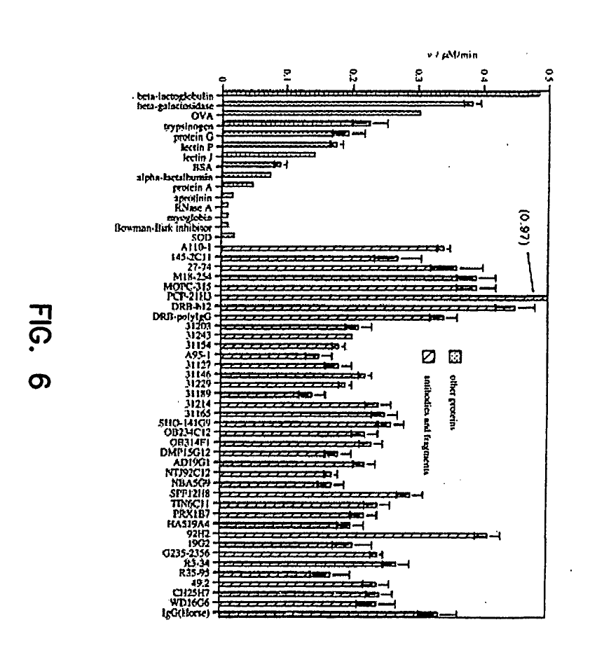

The mechanism by which antibodies reduce ¹O₂ is still being determined. However, the participation of a metal-mediated redox process has been largely discounted because the activity of the antibodies remains unchanged after exhaustive dialysis in PBS containing EDTA (4 mM). This leaves the intrinsic ability of the amino acid composition of the antibodies themselves. Aromatic amino acids such as tryptophan (Trp) can be oxidized by ¹O₂ via electron transfer (Grossweiner, L. I., Curr. Top. Radiat. Res. 11, 141-199 (1976)). In addition, disulfides are sufficiently electron rich that they can also be oxidized (Bent. D. V. & Hayon, E., J. Am. Chem. Soc., 87, 2612-2619 (1975)). Therefore, there is the potential that Trp residues and/or the intrachain or interchain disulfide bonds homologous to all antibodies are responsible for ¹O₂ reduction. To both investigate to what extent this ability of antibodies is shared by other proteins and to probe the mechanism of reduction, a panel of other proteins was studied (FIG. 6).

Whereas other proteins can convert ¹O₂ into O₂^{●-} in contrast to antibodies it is by no means a universal property. RNase A and superoxide dismutase, which do not possess Trp residues but have several disulfide bonds, do not reduce ¹O₂. Similarly, the Bowman-Birk inhibitor protein (Voss, R.-H., Ermler, U., Essen, L.-O., Wenzl, G., Kim, Y.-M. & Flecker, P., Eur. J. Biochem. 242, 122-131 (1996); Baek, J. & Kim, S., Plant Physiol. 102, 687 (1993)) that has seven disulfide bonds and zero Trp residues does not reduce ¹O₂. In contrast, chick ovalbumin, which has only 2 Trp residues (Feldhoff, R. & Peters, T. J., Biochem. J. 159, 529-533 (1976)), is one of the most efficient proteins at reducing ¹O₂.

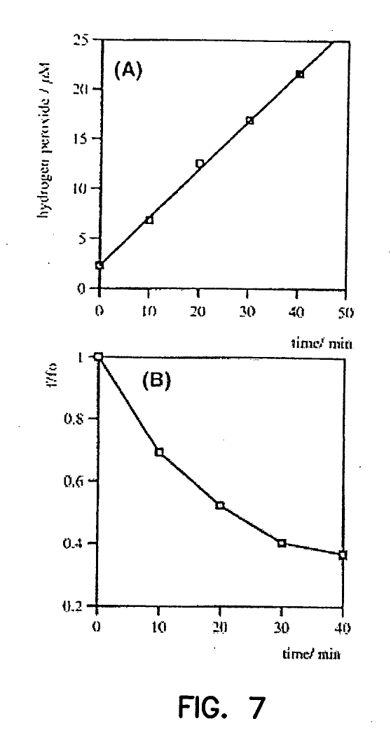

Given the loss of antibody activity upon denaturation, the location of key residues in the protein is likely to be more critical than their absolute number. Because the majority of aromatic residues in proteins are generally buried to facilitate structural stability (Burley, S. K. & Petsko, G. A., Science, 229, 23-28 (1985)), the nature of the reduction process was explored in terms of relative contribution of surface and buried residues by fluorescence-quenching experiments. Aromatic amino acids in proteins are modified by the absorption of ultraviolet light, especially in the presence of sensitizing agents such as molecular oxygen or ozone (Foote, C. S., Science 162, 963-970 (1968); Foote, C. S., Free Radicals Biol. 2, 85-133 (1976); Gollnick, KY., Adv. Photochem., 6, 1-122 (1968)). Trp reacts with 102 via a [2+2] cycloaddition to generate N-formylkynurenine or kynurenine, which are both known to significantly quench the emission of buried Trp residues (Mach, H., Burke, C. 3., Sanyal, G., Tsai, P.-K, Volkin, D. B. & Middaugh, C. R. in Formulation and Delivery of Proteins and Peptides, eds. Cleland, J. L. & Langer, R. (American Chemical Society, Denver, Colo.) (1994)). The intrinsic fluorescence of horse IgG is rapidly quenched to 30% of its original value during a 40 min irradiation, whereas hydrogen peroxide generation is linear throughout ([2=0.998) (FIG. 7). If the reduction of singlet oxygen is due to antibody Tip residues, then the solvent-exposed Trp seem to contribute to a lesser degree than the buried ones. This factor may help to explain why this ability is so highly conserved among antibodies. In greater than 99% of known antibodies there are two conserved Trp residues, and they are both deeply buried: TIP-36 and Trp-47 (Kabat, E. A., Wu, T. T., Perry, H. M., Gottesman, K. S. & Foeller, C., Sequences of Proteins of Immunological Interest (U.S. Department of Health and Human Services, Public Health Service, National Institutes of Health, Bethesda, Md.) (1991)).

Throughout nature, organisms have defended themselves by production of relatively simple chemicals. At the level of single molecules, this mechanism has thought to be largely abandoned with the appearance in vertebrates of the immune system. It was considered that once a targeting device had evolved, the killing mechanism moved elsewhere. The present results realign recognition with killing within the same molecule. In a certain sense this chemical immune system parallels the purely chemical defense mechanism of lower organisms, with the exception that a more sophisticated and diverse targeting element is added.

Given the constraints that an ideal killing system must use host molecules in a localized fashion while minimizing self damage, one can hardly imagine a more judicious choice than ¹O₂. Because one already has such a reactive molecule, it is important to ask what might be the advantage of its further conversion by the antibody. The key issue is that by conversion of the transient singlet oxygen molecule (lifetime 4 μs) into the more stable O₂^{●-} one now has access to hydrogen peroxide and all of the toxic products it can generate. In addition, superoxide is the only molecular oxygen equivalent remaining at the end of the oxygen-scavenging cascade. Therefore, this “recycling” may serve as a crucial mechanism for potentiation of the microbicidal process. Another benefit of singlet molecular oxygen is that it is only present when the host is under assault, thereby making it an “event-triggered” substrate. Also, because there are alternative ways to defend that use accessory systems, this chemical arm of the immune system might be silent under many circumstances. This said, however, there may be many disease states where antibody and singlet oxygen find themselves juxtaposed, thereby leading to cellular and tissue damage. Given that diverse events in man lead to the production of singlet oxygen, its activation by antibodies may lead to a variety of diseases ranging from autoimmunity to reperfusion injury and atherosclerosis (Skepper et al., Microsc. Res. Tech., 42, 369-385 (1998)).

### Example II

### Antibodies Catalyze the Oxidation of Water

### Methods and Materials

Crystalography: IgG 4C6 was digested with papain and the Fab′ fragment purified using standard protocols (Harlow and Lane). The Fab′ was crystallized from 13-18% PEG 8 K, 0.2 M ZnAc, 0.1 M cacodylate, pH 6.5. Crystals were pressurized under xenon gas at 200 psi for two minutes (Soltis et al., J. Appl. Cryst. 30, 190, (1997)) and then flash cooled in liquid nitrogen. Data were collected to 2.0 Å resolution at SSRL BL9-2. The structure was solved by molecular replacement using coordinates from the native 4C6 structure, and xenon atom sites were identified from strong peaks in the difference Fourier map. Refinement of the structure was done in CNS (Brünger et al., Acta. Crystallogr., D54, 905 (1998)) to final R=23.1% and R_{free}25.7%. The occupancies of the two xenon atoms were refined after fixing their B values fifty percent higher than the B factors of the immediately surrounding protein. The figures were generated in Bobscript (R. M. Esnouf, Acta Crystallog., D55, 938 (1999)).

Scanning of the Kabat database: The Kabat database of human and mouse sequences was analyzed to determine the number of Trp, Tyr, Cys, Met in their structures. Sequences were rejected if there were too many residue deletions or missing fragments. This allowed a high certainty analysis for 2068 of the 3894 sequences available. The values are reported as the mean totals with the range in parentheses of the C_{H}, V_{H}, C_{L}and V_{L} (κ and λ.) regions: Trp 15.5 (14 to 31), Tyr 30.4 (13 to 47), Cys 19.3 (15 to 29), Met 11.6 (7 to 32), His 13.3 (8 to 28). Grand total=90.1 (49 to 167).

Inductively coupled plasma atomic emission spectroscopy: Inductively coupled plasma atomic emission spectroscopy (ICP-AES) of mAb PCP21H3 was performed on a Varian, Axial Vista Simultaneous ICP-AES spectrometer. Mouse monoclonal antibody (PCP21H3) was exhaustively dialyzed into sodium phosphate buffered saline (PBS, 50 mM pH 7.4) with 20 mM EDTA. In a typical assay 300 μL of a 10.5% HNO₃ solution was added to 100 μL of a 10 mg/mL antibody solution and was incubated at 70° C. for 14 hours. This solution was then diluted to 2 mL with MQH₂O and then analyzed by comparison to standards. ICP-AES analysis results are reported in parts per million (μg/mL): Ag 0.0026 (0.0072 atoms per antibody molecule); A1 0.0098 (0.113 atoms per antibody molecule); As 0.0062 (0.025 atoms per antibody molecule); Ba below level of detection; Ca 0.0355 (0.266 atoms per antibody molecule). The high Ca concentration is a result of contamination of the phosphate buffer system utilized in our assay system. To investigate the effect of Ca(II) on the rate of antibody-mediated H₂O₂, the irradiation of antibody samples was performed using the assay procedure outlined in the legend of FIG. 8A with the addition of varying concentrations of CaCl₂ (0-100 μM). The process was found to be independent of Ca(II) concentration; Cd 0.0007 (0.0187 atoms per antibody molecule); Ce 0.0012 (0.003 atoms per antibody molecule); Co 0.0013 (0.007 atoms per antibody molecule); Cr 0.0010 (0.006 atoms per antibody molecule); Cu 0.0014 (0.007 atoms per antibody molecule); Fe 0.0089 (0.048 atoms per antibody molecule); Gd 0.0008 (0.001 atoms per antibody molecule); K 0.0394 (0.302 atoms per antibody molecule); La 0.0007 (0.002 atoms per antibody molecule); Li 0.0013 (0.056 atoms per antibody molecule); Mg 0.0027 (0.033 atoms per antibody molecule); Mn 0.0007 (0.004 atoms per antibody molecule); Mo 0.0023 (0.007 atoms per antibody molecule); Na 102.0428 (1332 atoms per antibody molecule); Ni 0.0007 (0.004 atoms per antibody molecule); P 14.3521 (138.9 atoms per antibody molecule); Pb below level of detection; Rb 0.0007 (0.002 atoms per antibody molecule); Se below level of detection; V 0.0109 (0.019 atoms per antibody molecule); W 0.0119 (0.019 atoms per antibody molecule); Zn 0.0087 (0.040 atoms per antibody molecule).

Oxygen isotope experiments: In a typical experiment, a solution of antibody (6.7 μM, 100 μL) or non-immunoglobulin protein (50 μM, 100 μL) in PB (160 mM phosphate; pH 7.4) was lyophilized to dryness and then dissolved in H₂O (100 μL, 98%). Sodium chloride was excluded to minimize signal suppression in the MS. 190 The higher concentration of non-immunoglobulin protein was necessary to generate a detectable amount of H2O₂ for the MS assay. This protein solution was irradiated on a UV-transilluminator under saturating ¹⁶O₂ aerobic conditions in a sealed quartz cuvette for 8 hours at 20° C. The H₂O₂ concentration was determined after 8 hours using the Amplex Red assay (Zhou et al., Anal. Biochem., 253, 162 (1997)). The sample was then filtered by centrifugation through a microcon (size-exclusion filter) to remove was the protein and the H₂O₂ concentration re-measured. TCEP (freshly prepared 20 mM stock in H₂¹⁸O) was added (ca. 2 mol eq relative to H₂O₂) and the solution was left to stand at 37° C. for 15 minutes, after which time all the H₂O₂ had reacted. The TCEP solution in H₂¹⁸O was prepared fresh prior to every assay because ¹⁸O is slowly incorporated into the carboxylic acids of TCEP (over days).

During the time course of the assay, no incorporation of ¹⁸O occurs due to this pathway. Furthermore, there is no incorporation of ¹⁸O from H₂¹⁸O into the ¹⁶O phosphine oxide. The peak at 249 m/z is the (M-H)⁻ of TCEP. The peak at 249 is observed in all the MS because an excess of TCEP (twofold) relative to H₂O₂ is used in the assay.

The reproducibility of the ¹⁶O/¹⁸O ratio from protein samples lyophilized together is reasonable (±10%). However, problems with removing protein-bound water molecules during the lyophilization process means that the observed ratios can vary between samples from different lyophilization batches by as much as 2:1 to 4:1 (when lyophilizing from H₂¹⁶O). It is, therefore, important that rigorous lyophilization and degassing procedures are followed. In this regard, the ¹⁸O₂ and H₂¹⁶O experiments exhibit far less inter-assay variability due to the relative ease of removing protein-bound oxygen molecules.

Antibodies from different species give similar ratios within the experimental constraints detailed below: ¹⁶O:¹⁸O: WD1-6G6 mIgG (murine) 2.1:1; polyIgG (horse) 2.2:1; polyIgG(sheep) 2.2:1; EP2-19G2 mIgG (murine) 2.1:1; CH2-5H7 mIgG (murine) 2.0:1; polyIgG (human) 2.1:1. Ratios are based on the mean value of duplicate determinations except for polyIgG (horse) which is the mean value of ten measurements. All assays and conditions are as described above.

In a typical experiment, a solution of sheep or horse polyIgG (6.7 μM, 100 μL) in PB (160 mM phosphate; pH 7.4) was degassed under an argon atmosphere for 30 min. This solution was then saturated with ¹⁸O₂ (90%) and irradiated as described above. Assays and procedures are then as described herein.

Assay for H2]production as a function of the efficiency of ¹O₂ formation via 302 sensitization with hematoporphyrin IX: The assay is a modification of a procedure developed by H. Sakai and co-workers, Proc. SPIE-Int. Soc. Opt. Eng. 2371, 264 (1995). In brief, the horse polyIgG (1 mg/mL) in PBS (50 mM, pH 7.4) and hematoporphyrin IX (40 μM) is irradiated with white light from a transilluminator. Aliquots are removed (50 μL) and the concentration of H₂O₂ and 3-aminophthalic acid measured simultaneously. H₂O₂ concentration was measured by the amplex red assay (Zhou et al., Anal. Biochem., 253, 162 (1997)). 3-Aminophthalic acid concentration was measured by reversed-phase HPLC on a Hitachi D4000 series machine with an Adsorbosphere-C18 column, a UV detector at 254 nm, and a mobile phase of acetonitrile/water (0.1% TFA) of 18:82 at 1 mL/min (retention time of luminol=7.4 min and 3-aminophthalic acid 3.5 min). The concentrations of luminol and 3-aminophthalic acid were determined by comparison of peak height and area to control samples. The experimental data yields the amount of ¹O₂ formed by hematoporphyrin IX (being directly proportional to the amount of 3-aminophthalic acid formed) and the amount of H₂O₂ formed by the antibody. N.B. There is no significant amount of ¹O₂ formed by antibodies without hematoporphyrin IX in white light.

Any concerns that the amplex red assay may be detecting protein-hydroperoxide derivatives in addition to H₂O₂ have been discounted because the apparent H₂O₂ concentration measured using this method is independent of whether irradiated protein is removed from the sample (by size-exclusion filtration).

Quantum Chemical Methods: All QC calculations were carried out with Jaguar [Jaguar 4.0, Schrödinger, Inc. Portland, Oreg., 1998. See B. H. Greeley, T. V. Russo, D. T. Mainz, R. A. Friesner, J.-M. Langlois, W. A. Goddard III, R. E. Donnelly, J. Chem. Phys. 101, 4028 (1994)] using the B3LYP flavor of density functional theory (DFT) [J. C. Slater in Quantum Theory of Molecules and Solids, Vol. 4: The Self-Consistent Field of Molecules and Solids, McGraw Hill, New York, (1974)], that includes the generalized gradient approximation and exact exchange. The 6-31G** basis set was used on all atoms. All geometries were fully optimized Vibrational frequencies were calculated to ensure that each minimum is a true local minimum (only positive frequencies) and that each transition state (TS) has only a single imaginary frequency (negative eigenvalue of the Hessian). Such QC calculations have been demonstrated to have an accuracy of 3 kcal/mol for simple organic molecules. Non-closed shell molecules such as O₂ and 302 are expected to have larger errors. However, such errors are expected to be systematic such that the mechanistic implications of the QC results should be correct. All energetics are reported in kcal/mol without correcting for zero point energy or temperature.

### Results and Discussion

Antibodies are capable of generating hydrogen peroxide (H₂O₂) from singlet molecular oxygen (¹O₂). However, it was not known until now, as reported herein, that the process was catalytic and the source of electrons. It is now shown that antibodies are unique as a class of proteins in that they can produce up to 500 mole equivalents of H₂O₂ from ¹O₂, without a reduction in rate, in the absence of any discernible cofactor and electron donor. Based on isotope incorporation experiments and kinetic data, it is proposed that antibodies are capable of facilitating an unprecedented addition of H₂O to ¹O₂ to form H₂O₃ as the first intermediate in a reaction cascade that eventually leads to H₂O₂. X-ray crystallographic studies with xenon point to conserved oxygen binding sites within the antibody fold where this chemistry could be initiated. This findings suggest a unique protective function of immunoglobulins against ¹O₂ and raise the question of whether the need to detoxify ¹O₂ has played a decisive role in the evolution of the immunoglobulin fold.

Antibodies, regardless of source or antigenic specificity, generate hydrogen peroxide (H₂O₂) from singlet molecular oxygen (¹O₂) thereby potentially aligning recognition and killing within the same molecule (Wentworth et al., Proc. Natl. Acad. Sci. U.S.A. 97, 10930 (2000)). Given the potential chemical and biological significance of this discovery, the mechanistic basis and structural location within the antibody of this process has been investigated. These combined studies reveal that in contrast to other proteins, antibodies may catalyze an unprecedented set of chemical reactions between water and singlet oxygen.

Kinetic studies. Long term UV irradiation studies reveal that antibody-mediated H₂O₂ production is a much more efficient process than is the case for the non-immunoglobulin proteins (FIG. 5A). Typically antibodies exhibit linearity in H₂O₂ formation for up to 40 mole equivalents of H₂O₂ before the rate begins to decline asymptotically (FIG. 8B). By contrast, non-immunoglobulin proteins display a short ‘burst’ of H₂O₂ production followed by quenching as photo-oxidation occurs (FIG. 8A).

In contrast to other proteins, antibodies are able to resume photo-production of H₂O₂ at the same initial rate as at the start of the experiment if the H₂O₂ generated during the assay is removed by catalase, as shown for murine monoclonal IgG PCP21H3 (FIG. 8C). This profile of continued linear production of H₂O₂ after catalase-mediated destruction of H₂O₂ was conserved for all antibodies assayed.

Thus, the H₂O₂ that accumulates during the process is inhibiting (reversibly) its own formation. The apparent IC₅₀ was estimated as 225 μM (FIG. 8D). Inhibition of the catalytic function of an enzyme either by substrates, transition state analogs or reaction products is often taken as strong evidence for an active site phenomenon. It has already been noted that the antibody-mediated photo-production of H₂O₂ is saturable with molecular oxygen (Kₘapp(O₂ 187 μM) (Wentworth et al., Proc. Natl. Acad. Sci. U.S.A., 97, 10930 (2000)). This formal product inhibition of H₂O₂ provides further evidence for such a binding site phenomenon.

An earlier report concerning the photo-production of H₂O₂ by antibodies did not probe the maximum amount of H₂O₂ that could be generated (Wentworth et al., Proc. Natl. Acad. Sci. U.S.A., 9, 10930 (2000)). This issue has been examined by repetitive cycles of UV irradiation of antibody samples followed by removal of the generated H₂O₂ by catalase (FIG. 8C shows two such cycles). In one series of experiments, the cycle of UV-irradiation and addition of catalase was carried out for up to 10 cycles (horse poly IgG in PBS, pH 7.4). During these experiments >500 mole equivalents (equiv.) of H₂O₂ were generated, with only a slight reduction in the initial rate being observed. Beside antibodies, the only other protein that was found thus far to generate H₂O₂ in such an efficient and long-term manner was the αβ T cell receptor (αβ TCR) (FIG. 8F). Interestingly, the αβ TCR shares a similar arrangement of its immunoglobulin fold domains with antibodies (Garcia et al., Science 274, 209 (1996)). However, possession of this structural motif seems not necessarily to confer an H₂O₂-generating ability on proteins as demonstrated by β₂-microglobulin which does not generate H₂O₂ even though it is a member of the immunoglobulin superfamily (Welinder et al., Mol. Immunol. 28, 177 (1991)).

The antibody structure is remarkably inert against the oxidizing effects of H₂O₂. When exposed to standard UV irradiation conditions for 6 hours in the presence of H₂O₂ (at a concentration high enough to fully inhibit H₂O₂ production), a polyclonal horse IgG antibody sample becomes fully active once the inhibitory H₂O₂ has been destroyed by catalase (FIG. 8E). The ability to continue H₂O₂ production for long periods at a constant rate, even after exposure to H₂O₂, reveals a remarkable, and hitherto unnoticed, resistance of the antibody structural fold to both chemical and photo-oxidative modifications suffered by other proteins. SDS-PAGE gel analysis of antibody samples after UV irradiation under standard conditions for 8 hours reveals neither significant fragmentation nor agglomeration of the antibody molecule. To ensure that there was no change in the protein structure in the presence of H₂O₂ (that may be contributing to the apparent inhibitory effect of H₂O₂) even at the level of side-chain position, x-ray crystal structures of Fab 4C6 were determined in the presence and absence of H₂O₂. Fab 4C6 was selected because its native crystals diffract to a higher resolution than any other published antibody (1.3 Å). The root mean square difference (RMSD) of key structural parameters were compared for the 4C6 structure before and after a soak experiment with 3 mM H₂O₂. RMSD of all atoms=0.412 Å, RMSD Cα atoms=0.327 Å, RMSD main chain atoms=0.328 Å, RMSD side-chain atoms=0.488 Å. The overlayed native and H₂O₂-treated structures of murine Fab 4C6 (Li et al., J. Am. Chem. Soc. 17, 3308 (1995)) are superimposable, reinforcing the evidence of stability of the antibody fold to H₂O₂ (FIG. 9).

An action spectrum of the antibody-mediated photo-production of H₂O₂ and the corresponding absorbance spectrum of the antibody protein for the same wavelength range (260-320 nm) are juxtaposed in FIG. 10. The two spectra are virtually superimposable with maximal efficiency of H₂O₂ production being observed at an excitation wavelength that coincides with the UV absorbance maxima of tryptophan in proteins.

Probing the efficiency of H₂O₂ production by horse IgG as a function of the efficiency of ¹O₂ formation via ³O₂ sensitization with hematoporphyrin IX (φ_{A}=0.22 in phosphate buffer pH 7.0 and visible light reveals that for every 275±25 mole equivalents of ¹O₂ generated by sensitization, 1 mole equivalent of H₂O₂ is generated by the antibody molecule (Wilkinson et al., J. Phys. Chem. Ref Data, 22, 113 (1993); Sakai et al., Proc. SPIE-Int. Soc. Opt. Eng. 2371, 264 (1995)).

The question of the electron source. The mechanism problem posed by the antibody-mediated H₂O₂ production from singlet oxygen has to be sharply divided into two sub-problems: one referring to the electron source for the process and the other concerning the chemical mechanism of the process. Given that the conversion of ¹O₂ to H₂O₂ requires two mole equivalents electrons, the fact that antibodies can generate >500 equivalents of H₂O₂ per equivalent of antibody molecule raises an acute electron inventory problem. The search for this electron source began with the most distinct possibilities. Since electron transfer through proteins can occur with remarkable facility and over notably large distances (Winkler et al., Pure & Appl. Chem., 71, 1753 (1999); Winkler, Curr. Opin. Chem. Biol., 4, 192 (2000)), the first considered was that a collection of the residues implicated as electron donors cited in normal protein photo-oxidation processes might be involved. The nearly constant rate of H₂O₂ production by antibodies and the αβ-TCR during the repetitive cycles of irradiation and catalase treatment (FIGS. 8C and 8E) argued against such a mechanism because a marked reduction of rate would have to accompany H₂O₂ production as the residues capable of being oxidized become exhausted. This reduction of rate would be further exacerbated because the redox potentials of the remaining unoxidized residues would have to rise as the protein becomes more positively charged.

Normal protein photo-oxidation is a complex cascade of processes that leads to the generation of ¹O₂ and other reactive oxygen species (ROS), such as superoxide anion (O₂^{●-}), peroxyl radical (HO₂^{●}) and H₂O₂ (Foote, Science 162.963(1968)). Present mechanistic thinking links the sensitivity of proteins to photo-oxidation with up to five amino acids: tryptophan (Trp), tyrosine (Tyr), cysteine (and cystine), methionine (et), and histidine (His) (Straight and Spikes, in Sin et O₂, A. A. Frimer, Ed. (CRC Press, Inc., Boca Raton, Fla., 1985), vol IV9, pp. 91-143; Michaeli and Feitelson, Photochem. Photobiol. 59, 284 (1994)). The photo-production of H₂O₂ by Trp and molecular oxygen is a well-characterized process that involves, at least in part, the formation and reduction of ¹O₂ to O₂ that spontaneously dismutates into H₂O₂ and ³O₂ (McMormick and Thompson, J. Am. Chem. Soc. 100, 312 (1978)). Tryptophan, both as an individual amino-acid and as a constituent of proteins, is particularly sensitive to near-UV irradiation (300-375 nm) under aerobic conditions, owing to its conversion to N′-formylkynurenine (NFK) that is a particularly effective near-UV (λ^{max} 320 nm) photosensitizer (Walrant and Santus, Photochem. Photobiol., 19, 411 (1974)). However, Trp photo-oxidation is accompanied by substoichiometric production of H₂O₂ (ca. 0.5 mole equivalents) during near-UV irradiation (FIG. 11A) (McMormick and Thompson, J. Am. Chem. Soc. 100, 312 (1978)) and the most efficient non-immunoglobulin protein at H₂O₂ photo-production, β-galactosidase, generates only 5.9 mol eq. of H₂O₂ from its 39 Trp residues FIG. 8A) (Fowler and Zabin, J. Biol. Chem., 253, 5521 (1978)).

Scanning of the Kabat database of human and mouse antibody heavy- and light-chain sequences (2068 of 3894 sequences were analyzed) revealed that antibodies rarely have more than 15 Trp residues in their entire structure (mean value=15.5 with a range of 14 to 31 Trp residues)(Kabat et al., Sequences of Proteins of Immunological Interest (US Department of Health and Human Services, Public Health Service, NIH, ed 5th, 1991); Martin, PROTEINS: Struct., Funct. and Genet., 25, 130 (1996)). In fact, even if all of the amino acids that are implicated in protein photo-oxidation processes vide supra are collectively involved in antibody-mediated H₂O₂-production, there is still an insufficient number of these residues (mean value=90.1 with a range of 49 to 167 reactive residues) to account for the 500 mole equivalents of H₂O₂ generated.

The potential of chloride ion (present at 150 mM in PBS) as a reducing equivalent was then investigated given that chloride ion is known to be a suitable electron source for photo-production of H₂O₂ via a triplet excited state of an anthraquinone (Scharf and Weitz, Symp. Quantum Chem. Biochem., Jerusalem vol. 12 (Catal. Chem. Biochem.: Theory Exp.), pp. 355-365 (1979)). This possibility was quickly discounted when the rate of H₂O₂ production by immunoglobulins was found to be independent of chloride ion concentration (FIG. 11B).

The possible role of metal ions was investigated. While such ions could hardly be present in antibodies in such amounts that they could serve as an electron source, trace amounts of them might play a central role as catalytic redox centers. Experiments were performed that, for all practical purposes, allow the implication of trace metals in this process to be ruled out The rate of antibody-mediated photo-production of H₂O₂ is unchanged before and after exhaustive dialysis of antibody samples with EDTA-containing buffer (FIG. 1I C). After EDTA treatment of antibody samples, ICP-atomic emission spectroscopy (AES) revealed the presence of trace metal ions remaining in amounts that are far below parts per million. For a trace metal to be implicated in this reaction it must be common to all antibodies because all antibodies assayed have this intrinsic ability. It is generally accepted that metal-binding is not an implicit feature of antibodies and is consistent with our own analysis of antibody crystals as well as the approximate 300 antibody structures available on the Brookhaven database.

All of the observations thus far forcibly pointed towards the need to identify an electron source that would not imply a deactivation of the protein catalyst and that could account for the high turnover numbers and hence, for a quasi unlimited source of electrons. A more broad consideration of the chemical potential of ¹O₂ was done. The participation of this energized form of molecular oxygen in the antibody-mediated mechanism was clearly inferred from a previous report (Wentworth et al., Proc. Natl. Acad. Sci. U.S.A., 97, 10930 (2000)). In brief, the antibody-mediated rate of H₂O₂ photo-production is increased in D₂O and reduced in the presence of the 102 quencher, sodium azide. Furthermore, antibodies have been shown to generate H₂O₂ via sensitization of 302 with hematoporphyrin IX in visible light, and in the dark with the endoperoxide of disodium 3′,3′-(1,4-naphthylidene) dipropionate (a chemical ¹O₂ source). The involvement of ¹O₂ is also in line with the close similarity of the action spectrum of antibody-mediated H₂O₂ production and the absorbance spectrum of antibody constituent tryptophans (FIG. 10).

Given that the known chemistry of ¹O₂ can be conceptualized as the chemistry of the super-electrophile “dioxa-ethene” (Foote, Acc. Chem. Res. 1, 104 (1968), the heretofore unknown possibility was considered that a molecule of water may, in the presence of an antibody, add as a nucleophile to ¹O₂ and form H₂O₃ as an intermediate. Thus, water becoming oxidized to H₂O₂ would fulfil the role of the electron source.

Oxygen isotope experiments were undertaken to test the hypothesis of an antibody-catalyzed photo-oxidation of H₂O by ¹O₂ through determination of the source of oxygen found in the H₂O₂. Contents of ¹⁶O/¹⁸O in H₂O₂ were measured by modification of a standard H₂O₂ detection method (Han et al., Anal. Biochem. 234, 107 (1996)). Briefly, this method involves reduction with tris carboxyethyl phosphine (TCEP), followed by mass-spectral (MS) analysis of the corresponding phosphine oxides (FIG. 12).

These experiments revealed that UV-irradiation of antibodies, in the presence of oxygen, leads to oxygen incorporation from water into H₂O₂ (FIGS. 12A and 12B). The relative abundance of the ¹⁶O/¹⁸O ratio observed in the MS of the phosphine oxide after irradiation of sheep polyIgG under conditions of saturating ¹⁶O₂ concentration in a solution of H₂¹⁸O (98% ¹⁸O) phosphate buffer (PB) is 2.2+0.2:1 (FIG. 12A). When the converse experiment is performed, with an ¹⁸O enriched molecular oxygen mixture (90% ¹⁸O) in H₂¹⁶O PB, the reverse ratio (1:2.0+0.2) is observed (FIG. 12B). These values of the ratios exhibit good reproducibility (+10 n=10) and are found for all antibodies studied.

The following control experiments were performed. First, under conditions of ¹⁶O₂ and H₂¹⁶O, irradiation of polyIgG Norse) generated H₂¹⁶O₂ (FIG. 12C). There is no incorporation of ¹⁸O when H₂O₂ (400 μM in PB, pH 7.0) itself is irradiated for 4 hours in H₂¹⁸O. This result alleviates concerns that ¹⁸O incorporation into H₂O₂ may be occurring via either an acid-catalyzed exchange with water or by a mechanism that involves homolytic cleavage of H₂¹⁶O₂ and recombination with H¹⁸O^{●} from water. To check the possibility that antibodies may catalyze both the production of H₂¹⁶O₂ and its acid-catalyzed exchange with H₂¹⁸O, the isotopic exchange of H₂¹⁶O₂ (200 μM) in H₂¹⁶O₂ (98% ¹⁸O) PB in the presence of sheep polyIgG (6.7 μM) after UV-irradiation under an inert atmosphere was determined. Only a trace of incorporation of 180 into H₂¹⁶O₂ was observed (FIG. 12D).

Isotope experiments were also performed with β-galactosidase, the most efficient non-immunoglobulin protein at generating H₂O₂, as well as 3-methylindole.

In both cases, photo-oxidation led to negligible ¹⁸O incorporation into the H₂O₂ (FIGS. 12E and 12F), illustrating the view that the indole ring itself and tryptophan residues in this protein are behaving simply as reductants of ¹O₂.

This view is further supported because irradiation of 3-methylindole generates H₂O₂ that does not include oxygen incorporation from H₂¹⁸O. The same experiment performed with tryptophan does give rise to exchange with a ratio ¹⁶O/¹⁸O 1.2:1. This result is thought to be due to the ammonium functionality acting as an intramolecular general acid, protonating the internal oxygen of a diastereomeric mixture of 3′-hydroperoxides (inset Below). It should be noted that while this is interesting from a chemical point of view, it cannot account for the catalytic production of H₂O₂ by antibodies both because it is a stoichiometric process and Trp residues in proteins do not possess a free ammonium group.

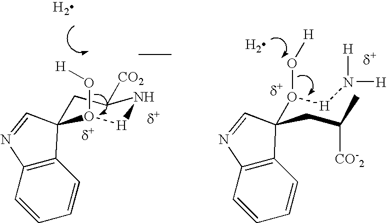

The chemical mechanism. All antibodies studied can catalyze the oxidation of water by singlet oxygen. The thermodynamic balance between reactants and products for the oxidation of H₂O by ¹O₂ (heat of reaction, ΔHᵣ°=+28.1 kcal/mol) (D. R. Lide, in Hanbook of Chemistry and Physics, 73rd ed. (CRC, 1992)), demands a stoichiometry in which more than one molecule of ¹O₂ would have to participate per molecule of oxidized water during its conversion into two molecules 6f H₂O₂. This stoichiometry assumes that no further light energy before that involved in the production of singlet from triplet oxygen is participating in the process. Qualitative chemical reasoning on hypothetical mechanistic pathways, together with thermodynamic considerations, makes the likely overall stoichiometries as in either equations 1b or c (all energetics are calculated from gas phase experimental heats of formation and are reported in kcal/mol):

¹O₂+2H₂O→2H₂O₂; ΔHᵣ°=28.1 (1a)

2¹O₂+2H₂O→2H₂O₂+³O₂; ΔHᵣ°=5.6 (1b)

3¹O₂+2H₂O→2H₂O₂+2³O₂; ΔHᵣ°=16.9 (1c)

A recent report of a transition metal-catalyzed conversion of ¹O₂ and water into hydrogen peroxide, via a tellurium-mediated redox process (Detty and Gibson, J. Am. Chem. Soc., 11, 4086 (1990)), provides experimental evidence for a process in which ¹O₂ and H₂O can be converted into 11202 and, hence that the energetic demands of this process can be overcome. It is thought that the mechanism for the antibody-mediated photo-oxidation process involves the addition of a molecule water to a molecule of ¹O₂ to form dihydrogen trioxide as the first intermediate on the way to H₂O₂. The antibody's function as a catalyst would have to be the supply of a specific molecular environment that would stabilize the critical intermediate relative to its reversible formation and, or, would accelerate the consumption of the intermediate by channeling its conversion to H₂O₂. An essential feature of such an environment might consist of a special constellation of organized water molecules at an active site conditioned by an antibody-specific surrounding.

While H₂O₃ has not yet been detected in biological systems, its chemistry in vivo has been a source of considerable speculation and its in vitro properties have been the subject of numerous experimental and theoretical treatments (C. Deby, La Recherche 228, 378 (1991); Sawyer, in Oxygen Chemistry (Oxford University Press, Oxford, 1991); Cerkovnik and Plesnicar, J. Am. Chem. Soc. 15, 12169 (1993); Vincent and Hillier, J. Phys. Chem. 99, 3109 (1995); Plesnicar et al., Chem. Eur. J., 809 (2000); Corey et al., J. Am. Chem. Soc. 108, 2472 (1986); Koller and Plesnicar, J. Am. Chem. Soc., 181, 2470 (1996); Cacace et al., Science 285, 81 (1999)). Plesnicar and co-workers have shown that H₂O₃, reductively generated from ozone, decomposes into H₂O and ¹O₂ (Koller and Plesnicar, J. Am. Chem. Soc., 118, 2470 (1996)). Applying the principle of microscopic reversibility, it was surmised that the reverse reaction should also be catalyzed by one or more molecules of water. To delineate plausible reaction routes and energetics of such a process, first principles quantum chemical (QC) methods were used (B3LYP Density Functional Theory) as described herein. The results are illustrated in equations 2a (all energetics are in kcal/mol):

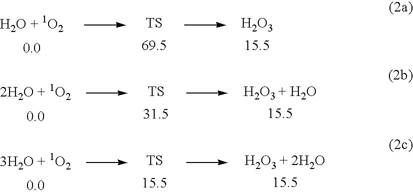

The direct reaction of water and ¹O₂ to give H₂O₃ is quite unfavorable, with an activation barrier of 70 kcal/mol (Eqn. 2a). However, with the addition of a second or third water molecule a concerted process is found that decreases the activation barrier to 31.5 and 15.5 kcal/mol respectively. Indeed these additional waters do play the role of a catalyst (in eqn. 2b the H of the 2nd water goes to the product HOOOH, simultaneous with the H of the 1st water replacing it). These barriers are small compared with the first HO bond energy of water (119 kcal/mol) and the bond energy of ¹O₂(96 kcal/mol). Note that the reverse reaction in eqn. 2b and eqn. 2c has a barrier of only 15.5 or 0 kcal/mol respectively, suggesting that H₂O₃ is not stable in bulk water or water rich systems. Thus, the best site within the antibody structure for producing and utilizing H₂O₃ is expected to be one in which there are localized waters and water dimers next to hydrophobic regions without such waters.

The ¹⁶O/¹⁸O ratio in the phosphine oxide derived from the antibody-catalyzed photo-oxidation of water poses a significant constraint to the selection of reaction paths by which this primary intermediate H₂O₃ would to convert to the final product H₂O₂. The ratio is primarily determined by the number of ¹O₂ molecules that chemically participate in the production of two moles of H₂O₂ from two moles of H₂O as well as by mechanistic details of this process. A ratio of 2.2:1 would coincide exactly with the value predicted for certain mechanisms in which two molecules of ¹O₂ and two molecules of H₂O are transformed into two molecules of H₂O₂ and one molecule of molecular oxygen (which would have to be ¹O₂ for thermodynamic reasons). An example of such a mechanism is an S_{H}2-type disproportionation of two molecules of H₂O₃ into H₂O₄ and H₂O₂, followed by the decomposition of the former into H₂O₂ and ³O₂. The complex problem of defining theoretically feasible reaction pathways for the conversion of H₂O₃ into H₂O₂ with or without the participation of ¹O₂ has been tackled in a systematic way using quantum chemical methods (933LYP Density Functional Theory). These studies show extensive docking calculations of H₂O₃ and the transition states for its formation and conversion into H₂O₂ to a number of proteins. Indeed there are unique sites of stabilizing these species in a region of antibodies (and the αβ-T cell receptor) in a region with isolated waters and next to hydrophobic regions. This extended study revealed the potential existence of a whole spectrum of theoretically feasible chemical pathways for the, H₂O₃ to H₂O₂ conversion.

Structural studies of xenon binding to antibodies. Given the conserved ability of antibodies, regardless of origin or antigen specificity, or of the αβ-TCR to mediate this reaction, X-ray structural studies were instigated to search for a possible conserved reaction site within these immunoglobulin fold proteins. A key constraint for any potential locus is that molecular oxygen (either ¹O₂ or triplet with a potential sensitizing residue in proximity, preferably tryptophan) and water must be able to co-localize, and the transition-states and intermediates along the pathway must be stabilized either within the site or in close proximity.

There is strong evidence to support the notion that Xe and O₂ co-localize in the same cavities within proteins (Tilson et al., J. Mol. Biol., 199, 195 (1988); Schoenborn et al., Nature, 207, 28 (1965)). Accordingly, xenon gas was used as a heavy atom tracer to locate cavities within the murine monoclonal antibody 4C6 that may be accessible to molecular oxygen (Li et al., J. Am. Chem. Soc., 117, 3308 (1995)).

Three xenon sites were identified (FIG. 13A), and all occupy hydrophobic cavities as observed in other Xe-binding sites in proteins (Scott and Gibson, Biochemistry 36, 11909 (1997); Prangé et al., PROTEINS: Struct. Funct. and Genet., 30, 61 (1998)). Superposition of the refined native and Xe-derivatized structures shows that, aside from addition of xenon, there is little discernible change in the protein backbone or side chain conformation or in the location of bound water molecules.

The xenon I binding site (Xe1 site) has been analyzed here in more detail because it is conserved in all antibodies and the αβ TCR (FIG. 13B). Xe1 is in the middle of a highly conserved region between the β-sheets of V_{L}, 7 Å from an invariant Trp. The Xe1 site is sandwiched between the two β-sheets that comprise the immunoglobulin fold of the V_{L}, approximately 5 Å from the outside molecular surface. Xenon site two (Xe2) sits at the base of the antigen binding pocket directly above several highly conserved residues that form the structurally conserved interface between the heavy and light chains of an antibody (FIG. 13A). The residues in the V_{L} V_{H} interface are primarily hydrophobic and include conserved aromatic side chains, such as Trp^{H109}.

The contacting side chains for Xe1 in Fab 4C6 are Ala^{L19}, Ile^{L21}, Leu^{L73}, and Ile^{L75}, which are highly conserved aliphatic side chains in all antibodies (Kabat et al., Sequences of Proteins of Immunological Interest (US Department of Health and Human Services, Public Health Service, NIH, ed. 5th, 1991)). Additionally, only slight structural variation was observed in this region in all antibodies surveyed. Notably, several other highly conserved and invariant residues are in the immediate vicinity of this xenon site, including Trp^{L35}, Phe^{L62}, Tyr^{L86}, Leu^{L104}, and the disulfide-bridge between Cys^{L23} and Cys_{L88}. Trp^{L35} stacks against the disulfide-bridge and is only 7 Å from the xenon atom. In this structural context, Trp^{L35} may be a putative molecular oxygen sensitizer, since it is the closest Trp to Xe1. Comparison with the 2C αβ TCR structure and all available TCR sequences shows that this Xe1 hydrophobic pocket is also highly conserved in TCRs (FIG. 5B) (Garcia, Science 274, 209 (1996)).

Human β₂-microglobulin, which does not generate H₂O₂, does not have the same detailed structural characteristics that define the antibody Xe1 binding pocket, despite its overall immunoglobulin fold. Also, β₂-microglobulin does not contain the conserved Trp residue that occurs there in both antibodies and TCRs. If Trp^{L35} (antibodies) or Trp^{α34} (TCR) is the oxygen sensitizer, the lack of a corresponding Tip in β₂-microglobulin may relate to the finding that it does not catalyze the oxidation of water.

Thus, the xenon experiments have identified at least one site that is both accessible to molecular oxygen and is in a conserved region (V_{L}) in close proximity to an invariant Trp; an equivalent conserved site is also possible in the fold of V_{H} The structure and sequence around the Xe1 site is almost exactly reproduced in the V_{H} domain by the pseudo two-fold rotation axis that relates V_{L} to V_{H}. Although a xenon binding-site was not located in this domain, it is thought that molecular oxygen can still access the corresponding cavity in V_{H}. The proposed heavy chain xenon site may not have been found because the crystals were pressurized for only two minutes, which may have been insufficient time to establish full equilibrium, or simply because xenon is too large compared to oxygen for the corresponding cavity on the V_{H} side, or due to crystal packing. In other antibody experiments, Xe binding sites were found in only one of the two molecules of the asymmetric unit that suggests that crystal packing can modulate access of Xe in crystals. Analysis of the sequence and structure around these sites shows that they are highly conserved in both antibodies and TCRs thus providing a possible understanding of why the Ig-fold in antibodies and the TCR can be involved in this unusual chemistry.

Antibodies are unique among proteins in their ability to catalytically convert ¹O₂ into H₂O₂. It is thought that this process participates in killing by event-related production of H₂O₂. Alternatively, antibodies can fulfill the function of defending an organism against ¹O₂. This would require the further processing of hydrogen peroxide into water and triplet oxygen by catalase.

### Publications

Burton, D. R., Trends Biochem. Sci., 5, 6469 (1990).

Arlaud, G. J., Colomb, M. G. & Gagon, J., Immunol. Today 8, 106-111 (1987).

Sim, R. B. & Reid, K. B., Immunol. Today 12, 307-311 (1991).

Wentworth, P., Jr. & Janda, K D., Curr. Opin. Chem. Biol., 2, 138-144 (1998).

Klebanoff, S. J. in Encyclopedia of Immunology. eds. Delves, P. J. & Roitt, I. M. (Academic, San Diego), pp. 1713-1718 (1998).

Rosen, H. & Klebanoff, S. J., J. Biol. Chem. 252, 48034810 (1997).

Fee, J. A. in International Conference on Oxygen and Oxygen-Radicals, eds. Rodgers, M. A. J. & Powers, E. L. (Academic, San Diego, and University of Texas at Austin), pp. 205-239 (1981).

Kearns, D. R., Chem. Rev., 1, 395427 (1971).

Foote, C. S. in Free Radicals in Biology ed. Pryor, W. A. (Academic, New York), pp. 85-133 (1976).

Allen, R C., Stjernholm, R L., Benerito, R. IL & Steele, R. H., eds. Cormier, M. J.,

Hercules, D. M. & Lee, J. (Plenum, New York), pp. 498-499 (1973).

Klebanoff, S. J. in The Phagocytic Cell in Host Resistance (National Institute of Child Health and Human Development, Orlando, Fla.) (1974).

Stauff, J., Sander, U. & Jaeschke, W., Chemiluminescence and Bioluminescence, eds., Williams, R. C. & Fudenberg, H. H. (Intercontinental Medical Book Corp., New York), pp. 131-141 (1973).

Allen, R. C., Yevich, S. J., Orth, R. W. & Steele, R H., Biochem. Biophys. Res. Commun. 60, 909-917 (1974).

Zhou, M., Diwu, Z., Panchuk-Voloshina, N. & Haugland, R P., Anal. Biochem., 253, 162-168 (1997).

Petyaev, I. M. & Hunt, J. V., Redox Report 2, 365-372 (1996).

Kreitner, M., Alth, G., Koren, H., Loew, S. & Ebermann, R., Anal. Biochem. 213. 63-67 (1993).

Beauchamp, C. & Fridovich, I., Anal. Biochem. 44, 276-287 (1971).

Srinivasan, V. S., Podolski, D., Westrick, N. J. & Neckers, D. C., J. Am. Chem. Soc. 100, 6513-6515 (1978).

Hasty, N., Merkel, P. B., Radlick, P. & Keams, D. R. Tetrahedron left., 49-52 (1972).

Merkel, P. B., Nillson, K & Keams, D. R., J. Am. Chem. Soc. 94, 1030-1031 (1972).

Grossweiner, L. I., Curr. Top. Radiat. Res. O. 11, 141-199 (1976).

Bent. D. V. & Hayon, E., J. Am. Chem. Soc. 87, 2612-2619 (1975). Voss, R.-H., Ermler, U., Essen, L.-O., Wenzl, G., Kim, Y.-M. & Flecker, P., Eur. J. Biochem., 242, 122-131 (1996).

Baek, J. & Kim, S., Plant Physiol., 10, 687 (1993).

Feldhoff, R. & Peters, T. J., Biochem. J. 159, 529-533 (1976).

Burley, S. K & Petsko, G. A., Science, 229, 23-28 (1985). Gollnick, K., Adv. Photochem. 6, 1-122 (1968). Mach, H., Burke, C. J., Sanyal, G., Tsai, P.-K, Volkin, D. B. & Middaugh, C. R. in Formulation and Delivery of Proteins and Peptides eds. Cleland, 3. L. & Langer, R. (American Chemical Society, Denver, Colo.) (1994).

Skepper, J., Pierson, R., Young, V., Rees, J., Powell, J., Navaratnam, V., Cary, N., Tew, D., Bacon, P., Wallwork, J. et al., Microsc. Res. Tech. 42, 369-385 (1998).

A. D. Wentworth, L. H Jones, P. Wentworth, Jr., K. D. Janda, R. A. Lerner, Proc. Natl. Acad. Sci. U.S.A., 97, 10930 (2000).

M. Zhou, Z. Diwu, N. Panchuk-Voloshina, R. P. Haugland, Anal. Biochem., 253, 162 (1997).

K C. Garcia et al., Science, 274, 209 (1996).

K G. Welinder, H. M. Jespersen, J. W.-Rasmussen, K Skoedt, Mol. Immunol 28, 177 (1991).

T. Li, S. Hilton, K. D. Janda, J. Am. Chem. Soc. 117.3308(1995).

F. Wilkinson, W. P. Helman, A. B. Ross, J. Phys. Chem. Ref Data 22, 113 (1993).

J. R. Winkler, A. J. Di Bilio, N. A. Farrow, J. H. Richards, H. B. Gray, Pure & Appl. Chem., 71, 1753 (1999).

J. R. Winkler, Curr. Opin. Chem. Biol., 4, 192 (2000).

C. S. Foote, Science 162, 963 (1968).

R. C. Straight, J. D. Spikes, in Singlet °, A. A. Frimer, Ed. (CRC Press, Inc., Boca Raton, Fla., 1985), vol. IV9, pp. 91-143.

A. Michaeli, J. Feitelson, Photochem. Photobiol. 59, 284 (1994).

J. P. McCormick, T. Thomason, J. Am. Chem. Soc. 100, 312 (1978).

P. Walrant, R. Santus, Photochem. Photobiol., 19, 411 (1974).

A. V. Fowler, I. Zabin, J. Biol. Chem. 253, 5521 (1978).

E. A. Kabat, T. T. Wu, H. M. Perry, K. S. Gottesman, C. Foeller, Sequences of Proteins of Immunological Interest (US Department of Health and Human Services, Public Health Service, NIH, ed. 5th, 1991).

A. C. R. Martin, PROTEINS: Struct. Funct and Genet., 60, 130 (1996).

H. D. Scharf, R Weitz, Symp. Quantum Chem. Biochem., Jerusalem vol. 12 (Catal. Chem. Biochem.: Theory Exp.), pp. 355-365 (1979).

C. S. Foote, Acc. Chem. Res., 1, 104 (1968).

J. Han, S. Yen, G. Han, P. Han, Anal. Biochem. 234, 107 (1996).

D. R Lide, in Handbook of Chemistry and Physics 73rd ed (CRC, 1992).

M. Detty, S. L. Gibson, J. Am. Chem. Soc. 112, 4086 (1990).

C. Deby, La Recherche 228, 378 (1991).

D. T. Sawyer, in Oxygen Chemistry (Oxford University Press, Oxford, 1991).

J. Cerkovnik, B. Plesnicar, J. Am. Chem. Soc., 115, 12169 (1993).

M. A. Vincent, I. A. Hillier, J. Phys. Chem. 99, 3109 (1995).

B. Plesnicar, J. Cerkovnik, T. Tekavec, J. Koller, Chem. Eur. J., 6, 809 (2000).

E. J. Corey, Mehrotra, M. M.; Khan, A. U., J. Am. Chem. Soc., 108, 2472 (1986).

J. Koller, B. Plesnicar, J. Am. Chem. Soc. 118, 2470 (1996).

F. Cacace, G. de Petris, F. Pepi, A. Troiani, Science 28, 81 (1999).

B. H. Greeley, T. V. Russo, D. T. Mainz, P, A. Friesner, J.-M. Langlois, W. A.

Goddard m, P E. Donnelly, J. Chem. Phys. 101, 4028 (1994)

J. C. Slater in Quantum Theory of Molecules and Solids, Vol. 4: The Self-Consistent Field of Molecules and Solids, McGraw Hill, New York, (1974)

R. F. Tilson Jr., U. C. Singh, I. D. Kuntz Jr. P. A. Kollman, J. Mol. Biol., 199, 195 (1988).

B. P. Schoenbom. H. C. Watson, J. C. Kendrew, Nature 207, 28(1965).

E. E. Scott, Q. H. Gibson, Biochemistry 36, 11909 (1997).

T. Prangé et al., PROTEINS: Struct. Funct. and Genet. 30, 61 (1998).

S. M. Soltis, M. A. B. Stowell. M. C. Wiener, G. N. Phillips Jr, D. C. Rees, J. Appl. Cryst., 30, 190, (1997)

A. T. Brünger et al., Acta. Crystallogr., D54, 905 (1998)

R. M. Esnouf, Acta Crystallog., D55, 938 (1999)].

J. R. Kanowsky, Chem. Biol. Interactions 70, 1 (1989).

V. Cannac-Caffrey, et al., Biochimie. 80, 1003 (1998).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details descried herein may be varied considerably without departing from the basic principles of the invention.

What is claimed is:

1. A method of treating a cell comprising contacting the cell with an antioxidant, wherein the antioxidant is effective in reducing antibody mediated generation of superoxide or hydrogen peroxide in the cell.

2. The method of claim 1 wherein the antioxidant is ascorbic acid, α-tocopherol, γ-glutamylcysteinylglycine, γ-glutamyl transpeptidase, α-lipoic acid, dihydrolipoate, N-acetyl-5-methoxysyptamine, flavones, flavonenes, flavanols, catalase, peroxidase, superoxide dismutase, metallothionein, or butylated hydroxytoluene.

3. The method of claim 1 wherein the antioxidant is contained in a liposome.

4. The method of claim 1 wherein the cell is an endothelial, interstitial, epithelial, muscle, phagocytic, white blood cells, dendritic, connective tissue or nervous system cell.

5. The method of claim 4 wherein the phagocytic cell is a neutrophil or a macrophage.

6. The method of claim 4 wherein the muscle cell is a smooth muscle cell, a skeletal muscle cell or a cardiac muscle cell.

7. A method of treating a subject comprising administering an antioxidant in a pharmaceutically acceptable excipient to the subject, wherein the antioxidant is effective in reducing antibody mediated generation of superoxide or hydrogen peroxide in a cell in the subject.

8. The method of claim 7, wherein the antibody mediated generation of superoxide or hydrogen peroxide causes oxidative stress in the subject.

9. The method of claim 8 wherein the oxidative stress is present in a subject presenting with disease conditions for cancer, inflammatory diseases, ischemic diseases, hemochromatosis, acquired immunodeficiency syndrome, emphysema, organ transplantation, gastric ulcers, hypertension, preeclampsia, neurological diseases, alcoholism and smoking-related diseases.

10. The method of claim 9 wherein the inflammatory diseases are arthritis, vasculitis, glomerulonephritis, systemic lupus erythematosus, and adult respiratory distress syndrome.

11. The method of claim 9 wherein the ischemic diseases are heart disease, stroke, intestinal ischemia, and reperfusion injury.

12. The method of claim 9 wherein the neurological diseases are multiple sclerosis, Alzheimer's disease, Parkinson's disease, amyotrophic lateral sclerosis, and muscular dystrophy.

13. The method of claim 7, wherein the antibody mediated generation of superoxide or hydrogen peroxide causes tissue injury in the subject.

14. The method of claim 13 wherein the tissue is selected from the group consisting of muscle, nervous, skin, glandular, mesenchymal, splenic, sclerous, epithelial and endothelial tissues.

15. The method of claim 7, wherein the antibody mediated generation of superoxide or hydrogen peroxide is associated with an inflammatory condition in the subject.

16. The method of claim 15 wherein the inflammatory condition is an inflammation of the lungs.

17. The method of claim 7, wherein the antibody mediated generation of superoxide or hydrogen peroxide is associated with a disorder resulting from aberrant smooth muscle function.

18. The method of claim 17 wherein the aberrant smooth muscle function is in the lung airways or vasculature.

19. The method of claim 7, wherein the antibody mediated generation of superoxide or hydrogen peroxide is associated with organ transplantation in the subject.

20. The method of claim 7 wherein the antioxidant is selected from the group consisting of ascorbic acid, α-tocopherol, γ-glutamylcysteinylglycine, γ-glutamyl transpeptidase, α-lipoic acid, dihydrolipoate, -acetyl-5-methoxytryptamine, flavones, flavonenes, flavanols, catalase, peroxidase, superoxide dismutase, metallothionein, and butylated hydroxytoluene.

21. The method of claim 7 wherein the composition is delivered to the subject intravenously, topically, orally, by inhalation, by cannulation, intracavitally, intramuscularly, transdermally, and subcutaneously.

22. The method of claim 7 wherein the composition comprises liposome containing the antioxidant.

23. A method for exposing an antigen to superoxide or hydrogen peroxide comprising contacting the antigen with an antibody capable of generating superoxide or hydrogen peroxide from singlet oxygen.

24. The method of claim 23 wherein singlet oxygen is induced with a sensitizer.

25. The method of claim 24 wherein the sensitizer is conjugated to the antibody.

26. The method of claim 25 wherein the sensitizer is selected from the group consisting of pterins, flavins, hematoporphyrin, tetrakis(4-sulfonatophenyl)porphyrin, bipyridyl ruthemium(II) complexes, rose bengal dye, quinones, rhodamine dyes, phtalocyanine, and hypocrellins.

27. The method of claim 23 wherein the antigen is a fatty acid or a low density lipoprotein.

28. The method of claim 23 wherein the antigen is presented on a cell.

29. The method of claim 23 wherein the cell is an endothelial, interstitial, epithelial, muscle, phagocytic, white blood cells, dendritic, connective tissue or nervous system cell.

30. The method of claim 29 wherein the phagocytic cell is a neutrophil or a macrophage.

31. The method of claim 29 wherein the muscle cell is a smooth muscle cell, a skeletal muscle cell or a cardiac muscle cell.

32. The method of claim 23 wherein the singlet oxygen is generated from irradiation of the cell.

33. The method of claim 32 wherein the irradiation of the cell is with ultraviolet light, infrared light or visible light.

34. The method of claim 23 wherein the antibody is a Fab, Fv, sFv or complete immunoglobulin molecule.

35. The method of claim 23 wherein the antibody is immunospecific for the antigen.

36. The method of claims 23 wherein the antibody is not immunospecific for the antigen.

37. The method of claim 23 wherein the antibody concentration at the cell surface is from 1-5 micromolar.

38. A method for inhibiting proliferation a cancer cell comprising contacting the cancer cell with a composition comprising an effective proliferation-inhibiting amount of antibody capable of generating superoxide or hydrogen peroxide from singlet oxygen.

39. The method of claim 38, wherein the amount of antibody is sufficient to kill the cancer cell.

40. The method of claim 38 wherein the antibody concentration at the cancer cell surface is from 1-5 micromolar.

41. The method of claim 38 wherein the antibody is a Fab, Fv, sFv or complete immunoglobulin molecule.

42. The method of claim 38 wherein the antibody recognize and immunoreacts with an antigen expressed on the cancer cell.

43. The method of claim 38 wherein the cancer cell is in a subject with cancer.

44. The method of claim 43 wherein the subject has lung cancer, prostate cancer, colon cancer, cervical cancer, endometrial cancer, bladder cancer, bone cancer, leukemia, lymphoma, or brain cancer.

45. The method of claim 43 wherein the cancer cell is removed from a subject with cancer and cultured ex vivo.

46. The method of claim 43 wherein the cell ex vivo is exposed to ultraviolet light, infrared light or visible light and is returned to the subject.

47. The method of claim 43 wherein the composition is delivered in vivo.

48. The method of claim 47 wherein the in vivo delivery is performed intravenously, topically, by inhalation, by cannulation, intracavitally, intramuscularly, transdermally, and subcutaneously.

49. The method of claim 38 wherein the composition comprises liposome containing the antibody.

50. The method of claim 49 wherein the antibody is a recombinant antibody.

51. The method of claim 50 wherein the recombinant antibody is expressed from an expression vector delivered to the cell.

52. The method of claim 51 wherein the expression vector further expresses a sensitizer molecule.

53. The method of claim 38 wherein the composition further comprises a sensitizer molecule.

54. The method of claim 53 wherein the sensitizer wherein the sensitizer molecule is selected from the group consisting of pterins, flavins, hematoporphyrin, tetrakis(4-sulfonatophenyl)porphyrin, bipyridyl ruthemium(II) complexes, rose bengal dye, quinones, rhodamine dyes, phtalocyanine, and hypocrellins.

55. The method of claim 53 wherein the sensitizer molecule is conjugated to the antibody.

56. The method of targeting and killing a cancer cell in a patient, the method comprising contacting the cancer cell with a composition comprising an effective killing amount of antibody in a pharmaceutically acceptable excipient, wherein the antibody is capable of generating superoxide or hydrogen peroxide from singlet oxygen, and wherein the antibody recognizes and immunoreacts with an antigen expressed on the cancer cell.

57. The method of claim 56 wherein the antibody concentration at the cell surface is from 1-5 micromolar.

58. The method of claim 56 further comprising placing the patient in a hyperbaric chamber.

59. The method of claim 56 wherein the composition further comprises a sensitizer molecule.

60. The method of claim 59 wherein the sensitizer wherein the sensitizer molecule is selected from the group consisting of pterins, flavins, hematoporphyrin, tetrakis(4-sulfonatophenyl)porphyrin, bipyridyl ruthemium(II) complexes, rose bengal dye, quinones, rhodamine dyes, phtalocyanine, and hypocrellins.

61. A method of treating a subject comprising administering to the subject a composition comprising a therapeutically effective amount of an antibody in a pharmaceutically acceptable excipient, wherein the antibody is capable of generating superoxide or hydrogen peroxide from singlet oxygen.

62. The method of claim 61, wherein the antibody mediated production of superoxide or hydrogen peroxide is associated with neutrophil mediated inflammation in the subject.

63. The method of claim 61, wherein the subject has an autoimmune disease.

64. The method of claim 61, wherein the antibody mediated production of superoxide or hydrogen peroxide enhances bactericidal effectiveness of a phagocyte in a subject.

65. The method of claim 61, wherein the antibody mediated production of superoxide or hydrogen peroxide promotes wound healing in a subject having a open wound.

66. The method of claim 65, wherein the superoxide or hydrogen peroxide stimulates fibroblast proliferation.

67. The method of claim 65, wherein the superoxide or hydrogen peroxide stimulates the immune response.

68. The method of claim 67, wherein the immune response includes lymphocyte proliferation.

69. The method of claim 61, wherein the antibody mediated production of superoxide or hydrogen peroxide stimulates cell proliferation.

70. The method of claim 69, wherein the cell population comprises fibroblasts in a wound in a subject.

71. The method of claim 70, wherein the cell population comprises lymphocytes in a wound on a subject.

72. The method of claim 71, wherein the lymphocytes comprise B cells.

73. The method of claim 69, wherein the contacting comprises topical application to a wound on a subject.

74. The method of claim 73, wherein topical application comprises a bandage containing the antibody.

75. A method for identifying an agent that modulates the production of hydrogen peroxide generated by antibody-mediated superoxide or hydrogen peroxide generation, the method comprising the steps of:
a) contacting a composition comprising an antibody capable of generating superoxide or hydrogen peroxide from singlet oxygen with the agent to form an admixture in an assay solution in the presence of molecular oxygen;
b) irradiating the admixture to generate singlet oxygen from molecular oxygen, wherein the singlet oxygen is reduced to hydrogen peroxide or superoxide by the antibody, wherein the superoxide dismutates to form hydrogen peroxide;
c) detecting the formed hydrogen peroxide; and
d) comparing the detected hydrogen peroxide with a suitable control, thereby determining how the agent modulates the production of hydrogen peroxide.

76. The method of claim 75, wherein the modulation is inhibition of hydrogen peroxide production.

77. The method of claim 75, wherein the modulation is generation of hydrogen peroxide production.

78. The method of claim 75, wherein the irradiation is with ultraviolet light.

79. The method of claim 75, wherein the irradiation is with visible light.

80. The method of claim 75, wherein the visible light irradiation further comprises admixing a sensitizer with the antibody composition.

81. The method of claim 75, wherein detecting the formed hydrogen peroxide is by fluorescent means with a fluorescent substrate for hydrogen peroxide.

82. The method of claim 75, wherein the fluorescent means are fluorescent microscopy or fluorescent spectrometry.

83. The method of claim 82, wherein the fluorescent spectrometry is ELISA based or with a standard cuvette.

84. The method of claim 75, wherein the steps are performed as described in example I.

85. A method for performing an immunoassay to detect antibody immunoreactivity with an antigen, the method comprising the steps of:
a) contacting in a singlet oxygen-generating medium a substrate having immobilized thereon a composition comprising a first reagent comprising an antigen or an antibody, with a second composition comprising an antigen or an antibody that is reactive with first reagent to form an immobilized antigen-antibody complex, wherein the antibody generates superoxide or hydrogen peroxide from singlet oxygen in the presence of oxygen; and
b) detecting the antibody-generated superoxide or hydrogen peroxide, thereby detecting the antibody immunoreactivity with the antigen.

86. The method of claim 85, further comprising irradiating the formed complex.

87. The method of claim 85, wherein the irradiation is with ultraviolet light.

88. The method of claim 85, wherein the irradiation is with visible light.

89. The method of claim 88, wherein the visible light irradiation further comprises admixing a sensitizer with the antibody.

90. The method of claim 85, wherein detecting the formed hydrogen peroxide is by fluorescent means with a fluorescent substrate for hydrogen peroxide.

91. The method of claim 90, wherein the fluorescent means are fluorescent microscopy or fluorescent spectrometry.

92. The method of claim 91, wherein the fluorescent spectrometry is ELISA based or with a standard cuvette.

93. The method of claim 85, wherein the first composition is an antigen and the second composition is an antibody.

94. The method of claim 85, wherein the first composition is an antibody and the second composition is an antigen.

95. The method of claim 85, wherein step (b) detects superoxide.

96. The method of claim 85, wherein step (b) detects hydrogen peroxide.

97. A therapeutic antioxidant comprising an engineered antibody molecule having less than two reductive centers, wherein production of superoxide or hydrogen peroxide from singlet oxygen reduced by the reductive center is diminished.

98. The therapeutic antioxidant of claim 97 further comprising a pharmaceutically acceptable excipient.

99. The therapeutic antioxidant of claim 97, wherein the antibody molecule is substantially free of a reductive center, wherein production of superoxide from singlet oxygen reduced by the reductive center is substantially absent.

100. The therapeutic antioxidant of claim 97, wherein the reductive center comprises indole.

101. The therapeutic antioxidant of claim 100, wherein the indole is present in an amino acid residue in the molecule.

102. The therapeutic antioxidant of claim 101, wherein the indole is present in a tryptophan residue.

103. The therapeutic antioxidant of claim 97, wherein the antibody is a recombinant antibody.

104. The therapeutic antioxidant of claim 97, wherein the antioxidant is used according to the method of claim 1 or 7.

105. The method of claim 1 or 7 wherein the antioxidant is the therapeutic antioxidant according to claim 97.

106. An engineered therapeutic molecule comprising greater than two reductive centers capable of reducing singlet oxygen to superoxide or hydrogen peroxide.

107. The engineered therapeutic molecule of claim 106 further comprising a pharmaceutically acceptable excipient.

108. The engineered therapeutic molecule of claim 106, wherein the reductive centers comprise indole.

109. The engineered therapeutic molecule of claim 106, wherein the molecule comprises amino acid residues.

110. The engineered therapeutic molecule of claim 109, where the indole is present in an amino acid residue in the molecule.

111. The engineered therapeutic molecule of claim 110, wherein the indole is present in a tryptophan residue.

112. The engineered therapeutic molecule of claim 111, wherein the tryptophan residue is present in an antibody.

113. The engineered therapeutic molecule of claim 106, wherein the antibody is a recombinant antibody.

114. The engineered therapeutic molecule of claim 113, wherein the recombinant antibody is capable of binding to an antigen.

115. The engineered therapeutic molecule of claim 113, wherein the recombinant antibody is expressed as a fusion conjugate.

116. The engineered therapeutic molecule of claim 115, wherein the fusion conjugate comprises a sensitizer.

117. The engineered therapeutic molecule of claim 112, wherein the tryptophan residue is present in ovalbumin.

118. The engineered therapeutic molecule of claim 106, wherein the molecule is chemically synthesized.

119. The engineered therapeutic molecule of claim 112, wherein the antibody is used according to the method of claim 7, 38 or 61.

120. The method of claim 7, 38 or 61 wherein the antibody is an engineered therapeutic antibody according to claim 106.

121. An engineered therapeutic antibody comprising at least one reductive center capable of reducing singlet oxygen to superoxide or hydrogen peroxide, and a pharmaceutically acceptable excipient.

122. The engineered therapeutic antibody of claim 121, wherein the reductive center comprises indole.

123. The engineered therapeutic antibody of claim 122, wherein the indole is present in an amino acid residue in the antibody.

124. The engineered therapeutic antibody of claim 123, wherein the indole is present in a tryptophan residue.

125. The engineered therapeutic antibody of claim 121, wherein the antibody is capable of binding to an antigen.

126. The engineered therapeutic antibody of claim 121, wherein the reductive center is positioned adjacent to a variable binding domain of the antibody.

127. The engineered therapeutic antibody of claim 121, wherein the antibody has three tryptophan residues.

128. The engineered therapeutic antibody of claim 121, wherein the antibody is a recombinant antibody.

129. The engineered therapeutic antibody of claim 128, wherein the recombinant antibody is expressed as a fusion conjugate.

130. The engineered therapeutic antibody of claim 129, wherein the recombinant antibody is capable of binding to an antigen and wherein the fusion conjugate comprises a sensitizer.

131. The engineered therapeutic antibody of claim 121, wherein the antibody is used according to the method of claim 7, 38 or 61.

132. The engineered therapeutic antibody of claim 125, wherein the antibody is used according to the method of claim 7, 38 or 61.

133. The engineered therapeutic antibody of claim 130, wherein the recombinant conjugated antibody is used according to the method of claim 86, 91, 93 or 94.

134. The method of claim 38 wherein the antibody is capable of binding to an antigen.

135. The method of claim 134, wherein singlet oxygen is produced by administering a prodrug that is capable of generating singlet oxygen, wherein the prodrug is administered after an appropriate time period to allow the antibody to bind to the antigen to form an antibody-antigen complex.

136. The method of claim 135, wherein the prodrug is endoperoxide.

137. The method of claim 136, wherein endoperoxide is present in a concentration of about 10 micromolar in proximity to the formed antibody-antigen complex.

138. The method of claim 135, wherein the antibody and the prodrug are administered intramuscularly, intravenously, or subcutaneously.

139. The method of claim 134, wherein the antibody is an engineered therapeutic antibody according to claim 121.

140. The method of claim 134 further comprising irradiation with ultraviolet light, infrared light or visible light, wherein the antibody is an engineered therapeutic antibody according to claim 121, and wherein the fusion conjugate comprises a sensitizer.

141. A method to detect the presence of an antigen in a bodily fluid comprising:
a) immobilizing a complex of the antigen with an antibody that is capable of generating superoxide or hydrogen peroxide; and
b) detecting the superoxide or hydrogen peroxide generated by the antibody.

142. The method of claim 141, wherein the antigen is a drug.

143. The method of claim 141, wherein the antigen is a hormone.

144. The method of claim 141, wherein the bodily fluid is blood or urine.

145. A composition comprising a T-cell receptor that can generate hydrogen peroxide.
